(19) United States
(12) Patent Application Publication
(10) Pub. No.: US 2019/0014791 A1
(43) Pub. Date: Jan. 17, 2019

(54) CANOLA OIL COMPOSITIONS WITH PARTICULAR TRIACYLGLYCEROL DISTRIBUTIONS

(71) Applicant: CARGILL, INCORPORATED, WAYZATA, MN (US)

(72) Inventor: DILIARA IASSONOVA, MAPLE GROVE, MN (US)

(21) Appl. No.: 15/749,341

(22) PCT Filed: Jul. 29, 2016
(86) PCT No.: PCT/US16/44719

§ 371 Date: Jan. 31, 2018

Related U.S. Application Data
(60) Provisional application No. 62/199,724, filed on Jul. 31, 2015.

(51) Int. Cl. A23D 9/013; A23D 9/02; A01H 5/10; A01H 1/04; A01H 6/20

(52) U.S. Cl. CPC A23D 9/013; A23D 9/02; A01H 6/202; A01H 1/04; A01H 5/10

(57) ABSTRACT

This application relates to canola oils comprising triacylglycerols (TAGs), wherein 11-16% of the total TAGs in the oil comprise one saturated fatty acid and two unsaturated fatty acids and wherein 82-88% of total TAGs comprise three unsaturated fatty acids, and wherein 81-91% of the total TAGs comprise at least one oleic acid and wherein 7-12% of the total TAGs comprise at least one linolenic acid. In some embodiments, the oils have a low saturated fatty acid content of 3.5-5.5%, such as 3.5-4.5%, or 3.5-4.0%.

### RELATED APPLICATIONS

This application claims priority to U.S. Provisional Patent Application No. 62/199,724, filed Jul. 31, 2015, and which is incorporated by reference in its entirety herein.

### FIELD

This application relates to canola oils having particular distributions of triacylglycerols (TAGs), uses of such oils, and Brassica plants producing the same.

### BACKGROUND

The fatty acid content of typical commodity type canola oils is about 6-8% total saturated fatty acids, about 55-65% oleic acid, about 22-30% linoleic acid, and about 7-10% linolenic acid. It may be helpful for some applications to reduce the saturated fatty acid and linolenic acid content of canola oils. For example, consumers may prefer oils with a reduced level of saturated fatty acid, while alpha-linolenic acid (ALA), for example, is unstable and easily oxidized during cooking and storage, which in turn creates off-flavors of the oil. Prior attempts to control the levels of oleic acid, linolenic acid, and saturated fatty acids in canola oils through genetic manipulation of Brassica plants are described, for example, in WO 2011/075716, WO 2011/150028, and WO 2015/077661, which are incorporated herein by reference.

The bulk of the fatty acids found in Brassica seeds are incorporated into triacylglycerols. Thus, it may also be beneficial to control the distribution of triacylglycerols in canola oils in order to provide canola oils with favorable properties for consumer use.

### SUMMARY

This application relates to canola oils having particular distributions of triacylglycerols (TAGs). TAGs are molecules comprising one glycerol moiety and three fatty acid moieties. In some embodiments, the oils have a TAG distribution such that 11-16%, such as 11-13%, of the total TAGs in the oil comprise one saturated fatty acid and two unsaturated fatty acids and wherein 82-88% of total TAGs comprise three unsaturated fatty acids. In some embodiments, 81-91% of the total TAGs comprise at least one oleic acid and wherein 7-12% of the total TAGs comprise at least one linolenic acid. In some such embodiments, there are no detectable TAGs comprising two or three saturated fatty acids.

In some embodiments, the canola oil further comprises 62-74% oleic acid and 2.5-5% linolenic acid. In some embodiments, the oil comprises 3.5% to 5.5% saturated fatty acid, such as 3.5% to 4.5% or 3.5% to 4.0%. In some embodiments, the oil comprises no more than 1% sterols, such as no more than 0.5% or no more than 0.4%. In some embodiments, the oil comprises no more than 0.5% trans fatty acids. In some embodiments, the oil comprises no more than 0.1% tocopherols or no more than 0.15% tocopherols.

In some embodiments, 3-5% of total TAGs comprise two oleic acids and one palmitic acid (designated POO). In some embodiments, 1-2% of total TAGs comprise two oleic acids and one stearic acid (designated SOO). In some embodiments, 1-3% of total TAGs comprise three linoleic acids (designated LLL). In some embodiments, 10-13% of total TAGs comprise one oleic acid and two linoleic acids (designated OLL).

In some embodiments, the oils comprise phospholipids and also have particular distributions of saturated phospholipids (PLs), the levels of saturated PLs may be similar to that found in higher saturated fat or wild-type oils. In some embodiments, 10-13% of the fatty acids found in the phosphatidyl choline fraction of the oil are saturated fatty acids. In some embodiments, 24-31% of the fatty acids found in the phosphatidyl ethanolamine fraction are saturated. In some embodiments, 17-30% of the fatty acids found in the phostphatidyl inositol fraction are saturated.

In some embodiments, the oil has been degummed, refined, bleached, dewaxed, and/or deodorized. In some embodiments, the oil has been emulsified or crystallized, such as to produce a semi-solid state, for example, for preparation of a margarine or shortening.

In some embodiments, the oil is produced from a Brassica plant line comprising one or more mutant alleles, such as one or more of the following: (a) a mutant allele at a fatty acyl-acyl carrier protein thioesterase A2 (FATA2) locus, wherein said mutant allele results in production of a FATA2 polypeptide having reduced thioesterase activity relative to a corresponding wild-type polypeptide, (b) a mutation at the chromosome N01 quantitative trait locus 1 (QTL1) allele and/or at the chromosome N19 quantitative trait locus 2 (QTL2) allele described in WO 2015/077661, incorporated herein by reference, (c) a mutant allele at a fatty acyl-acyl carrier protein thioesterase B (FATB) locus, such as any combination of mutant alleles at the FATB1, FATB2, FATB3, and FATB4 loci, wherein the mutant allele(s) results in production of a FATB polypeptide having reduced thioesterase activity relative to a corresponding wild-type FATB polypeptide, (d) a mutant allele at a delta-12 fatty acid desaturase (FAD2) locus, and (e) a mutant allele at a delta-15 fatty acid desaturase (FAD3) locus, such as at any combination of FAD3A, FAD3B, FAD3D, and FAD3F.

In some embodiments, the canola oils described herein may be produced from a Brassica plant that is a hybrid of the 03LC.034, Salomon, and mIMC201 breeding lines (described in Example 1). In some such embodiments, the plant is: (i) homozygous for mutant alleles in QTL1 of N01, QTL2 of N19, FATA2, FATB1 and FATB4, (ii) homozygous for mutant alleles in QTL1 of N01, QTL2 of N19, FATA2, and each of FATB1, 2, 3, and 4, or (iii) homozygous for mutant alleles in QTL1 of N01, QTL2 of N19, FATA2, and FATB3 and 4, and heterozygous for a mutant allele in FATB2. In other embodiments, the canola oils described herein may be produced from a Brassica plant that is a hybrid of the 07RFS43.001, Salomon, and IMC201 breeding lines, wherein the plant is: (i) homozygous for mutant alleles in QTL1 of N01, QTL2 of N19, FATA2, FATB1 and FATB4, (ii) homozygous for mutant alleles in QTL1 of N01, QTL2 of N19, FATA2, FATB3 and FATB4, or (iii) homozygous for mutant alleles in QTL1 of N01, QTL2 of N19, FATA2, and each of FATB1, 3, and 4.

In some embodiments, the Brassica plant from which the oil is produced is not a plant any of the Salomon (ATCC deposit no. PTA-11453), IMC201, 1764, 15.24, Skechers, or hybrid lines described in WO 2011/075716 and WO 2015/077661, incorporated by reference herein.

In some embodiments, the Brassica plant is herbicide tolerant. In some embodiments, the Brassica plant is non-transgenic, or is free of transgenes other than those for herbicide tolerance. In some embodiments, the Brassica plant has a yield that exceeds that of an open-pollinated spring canola variety such as 46A65 or Q2 by at least 10%, such as by 10-15%, 10-20%, 15-20%, 10-25%, 15-25%, 20-25%, or 10-35%. In some embodiments, the Brassica plant is a hybrid having a yield within 20%, such as within 15%, such as within 10%, such as within 5% of the yield of its highest yielding parental plant line.

Also comprised herein are methods of using the above oils to prepare food compositions such as a margarine or shortening as well as food compositions prepared from the above oils.

Further comprised herein are Brassica plants that produce the oils described above, and parts of such plants such as seeds, and progeny of such plants. Also comprised herein are methods of producing the oils above from such Brassica plants, for example, comprising pressing seeds of the plants to separate oil from seed hulls and extracting oil from the pressed seeds with hexane extraction and combining the separated oil and extracted oil fractions. Additional, optional steps include degumming, refining, bleaching, dewaxing, and/or deodorizing the oil.

It is to be understood that both the foregoing general description and the following more detailed description are exemplary and explanatory only and are not restrictive of the claims.

### BRIEF DESCRIPTION OF THE DRAWINGS

FIG. 1 shows the breeding scheme used to develop the breeding lines and samples tested in the examples of this application. As noted on the figure, the left, light-shaded box shows mIMC201 lineage; the right, darker-shaded box shows Salomon lineage, and the center, unshaded box shows Salomon and mIMC201 lineage, and MAS stands for marker assisted selection. (See Example 1 for further description.)

### DESCRIPTION OF PARTICULAR EMBODIMENTS

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Definitions of particular terms may be contained within this section or may be incorporated into the sections of text below.

In this application, the use of “or” means “and/or” unless stated otherwise. In the context of a multiple dependent claim, the use of “or” refers back to more than one preceding independent or dependent claim in the alternative only. Unless otherwise indicated, the term “include” has the same meaning as “include, but are not limited to,” the term “includes” has the same meaning as “includes, but is not limited to,” and the term “including” has the same meaning as “including, but not limited to.” Similarly, the term “such as” has the same meaning as the term “such as, but not limited to.” Also, terms such as “element” or “component” encompass both elements and components comprising one unit and elements and components that comprise more than one subunit unless specifically stated otherwise.

A “fatty acid” refers to a molecule comprising a hydrocarbon chain and a terminal carboxylic acid group. As used herein, the carboxylic acid group of the fatty acid may be modified or esterified, for example as occurs when the fatty acid is incorporated into a glyceride or a phospholipid or is attached to another molecule such as acetyl-CoA (e.g., COOR, where R refers to, for example, a carbon atom). Alternatively, the carboxylic acid group may be in the free fatty acid or salt form (i.e., COO⁻ or COOH).

A “saturated” fatty acid is a fatty acid that does not contain any carbon-carbon double bonds in the hydrocarbon chain. An “unsaturated” fatty acid contains one or more carbon-carbon double bonds. A “polyunsaturated” fatty acid contains more than one such carbon-carbon double bond while a “monounsaturated” fatty acid contains only one carbon-carbon double bond. Carbon-carbon double bonds may be in one of two stereoconfigurations denoted “cis” and “trans.” Naturally-occurring unsaturated fatty acids are generally in the “cis” form. Fatty acids in the “trans” form, which may be produced by partial hydrogenation of unsaturated fatty acids, may be potentially harmful to health.

Fatty acids found in plants and oils described herein may be incorporated into various glycerides. The terms “triacylglycerol,” “triglyceride,” and “TAG” are used interchangeably herein to refer to a molecule comprising a glycerol that is esterified at each of its three hydroxyl groups by a fatty acid and thus, comprises three fatty acids. The terms “diacylglycerol,” “diglyceride,” and “DAG” refer to a molecule comprising a glycerol esterified by a fatty acid at only two of its three available hydroxyl groups, such that it contains only two fatty acids Likewise, the term “monoglyceride” refers to a glycerol modified by a fatty acid at only one of the available three hydroxyl groups so that it comprises only one fatty acid.

Fatty acids found in plants and oils described herein may also be incorporated into various “phospholipids,” abbreviated “PL” herein. Phospholipids are molecules that comprise a diglyceride, a phosphate group, and another molecule such as choline (“phosphatidyl choline;” abbreviated “PC” herein), ethanolamine (“phosphatidyl ethanolamine;” abbreviated “PE” herein), serine “phosphatidyl serine;” abbreviated “PS” herein), or inositol (“phosphatidyl inositol;” abbreviated “PI” herein). Phospholipids, for example, are important components of cellular membranes.

Fatty acids in plants and oils may also be found in the “free fatty acid” form, meaning that the fatty acid (COO⁻ or COOH) group has not been esterified or otherwise covalently modified at the terminal carboxylic acid group.

Fatty acids described herein include those listed in the table below along with abbreviations used herein and structural formulae. According to Table 1 below, the naming convention comprises the number of carbons in the fatty acid chain (e.g. C16, C18, etc.) followed by a colon and then the number of carbon-carbon double bonds in the chain, i.e. 0 for a saturated fatty acid comprising no double bonds or 1, 2, 3, etc. for an unsaturated fatty acid comprising one, two, or three double bonds.

TABLE 1
Fatty acid nomenclature

| Fatty acid name (abbreviation) | Formula |
|---|---|
| Lauric acid (La) | C12:0 |
| Myristic acid (M) | C14:0 |
| Palmitic acid (P) | C16:0 |
| Palmitoleic acid (Po) | C16:1 |
| Stearic acid (S) | C18:0 |
| Oleic acid (O) | C18:1 |
| Linoleic acid (L) | C18:2 |
| Linolenic acid (Ln) | C18:3 |
| Arachinic acid (A) | C20:0 |
| Gondoic acid (G) | C20:1 |
| Behenic acid (B) | C22:0 |
| Erucic acid (E) | C22:1 |
| Lignoceric acid (Li) | C24:0 |

The levels of particular types of fatty acids or of types of TAGs or PLs may be provided herein in percentages. Unless specifically noted otherwise, such percentages are weight percentages based on the total fatty acids, TAGs, or PLs in the oil, respectively, as calculated experimentally. Thus, for example, if a percentage of a specific species or class of fatty acid is provided, e.g., oleic acid, this is a w/w percentage based on the total fatty acids detected in the oil. Similarly, if a percentage of a specific species or class of TAG is provided, this is a w/w percentage based on the total TAGs detected in the oil.

The term “sterol” or “sterols” as used herein refers to molecules comprising a steroid alcohol group, i.e. a steroid ring structure with a hydroxyl group at the 3-position of the A-ring. Examples include cholesterol, campesterol, sitosterol, and stigmasterol. If a particular percentage of sterols is provided herein, unless specifically noted otherwise, this is a w/w percentage based on the weight of the oil as calculated experimentally.

The term “tocopherol” or “tocopherols” as used herein refers collectively to alpha, beta, gamma, and delta-tocopherol. If a particular percentage of tocopherol is provided herein, unless specifically noted otherwise, this is a w/w percentage based on the weight of the oil as calculated experimentally.

The term “canola oil” as used herein refers to an oil derived from seeds or other parts of Brassica plants. In some embodiments, the oil also may be chemically treated or refined in various ways, for example by degumming, refining, bleaching, dewaxing, and/or deodorizing.

As used herein, reference to a Brassica “plant” or “plants” includes the plant and its progeny, such as its F₁, F₂, F₃, F₄, and subsequent generation plants. Brassica plants may include, for example B. napus, B. juncea, and B. rapa species. As used herein, a “line” or “breeding line” is a group of plants that display little or no genetic variation between individuals for at least one trait, such as a particular gene mutation or set of gene mutations. Such lines may be created by several generations of self-pollination and selection or by vegetative propagation from a single parent using tissue or cell culture techniques. A “variety” refers to a line that is used for commercial production and includes hybrid and open-pollinated varieties.

An “allele” refers to one or more alternative forms of a gene at a particular locus.

### Canola Oils

Several types of canola oil are currently commercially available containing, for example, different amounts of oleic (58-82%), linoleic (8-24%) and linolenic (1.5-11%) acids, for example, as well as <2% erucic acid and with saturated fatty acid contents of 6-8%.

This application relates to canola oils having particular distributions of TAGs. In some embodiments, the oils have a TAG distribution such that 11-16%, such as 11-13%, of the total TAGs in the oil comprise one saturated fatty acid and two unsaturated fatty acids and wherein 82-88% of total TAGs comprise three unsaturated fatty acids. The percentages of these two classes of TAGs are weight to weight (w/w) percentages based on determining the total TAG content of the oil and normalizing it to 100%. In some such embodiments, there are no detectable TAGs comprising two or three saturated fatty acids. In some embodiments, 81-91% of the total TAGs comprise at least one oleic acid and wherein 7-12% of the total TAGs comprise at least one linolenic acid. Such percentages may be obtained by determining the levels of each TAG species found in the oil, for example by chromatography methods as described in the Examples herein, normalizing the percentages to 100%, and then adding the percentages of each TAG species that contains one, two, or three oleic acid residues. For example, one may add together the percentage of the TAG species having three oleic acids (designated OOO herein based on the abbreviations shown in Table 1) to the percentage of the TAG species having one oleic acid and two linoleic acids (OLL herein), to the percentages of all of the other TAG species having one or two oleic acids, etc. Similarly, to determine the percentage of a given type of fatty acid in the total TAGs, one may add up the percentages of each TAG species that contains that fatty acid.

In some embodiments, 3-5% of total TAGs comprise two oleic acids and one palmitic acid (designated POO). In some embodiments, 1-2% of total TAGs comprise two oleic acids and one stearic acid (designated SOO). In some embodiments, 1-3.3% of total TAGs comprise three linoleic acids (designated LLL) such as 2.5-5% or 3.6-5%. In some embodiments, 9-14% of total TAGs comprise one oleic acid and two linoleic acids (designated OLL), such as 10-14% or 10-13%. In some embodiments, 2-5% of total TAGs comprise one oleic acid, one linolenic, and one linoleic acid (designated OLnL), such as 2.5-5% or 3.6-5%. These percentages may be obtained by determining the amounts of each TAG species in the oil, for example using a chromatography set-up as described in the Examples below. Once the amounts of each detectable TAG in the oil are determined, the results are normalized to 100% and given in percentages for each TAG species. Such an experiment cannot distinguish between TAG species in which individual fatty acids are located at the sn1, sn2, and sn3 positions of the glycerol. Thus, the amount of the TAG species denoted OLnL is a sum of the amounts of OLnL, OLLn, LOLn, LLnO, LnLO, and LnOL, for example, in which each O, L, and Ln fatty acid moiety is in each of the sn1, sn2, and sn3 positions on the glycerol.

In some embodiments, the canola oil further comprises 62-74% oleic acid and 2.5-5% linolenic acid. In some embodiments, the oil comprises 3.5% to 5.5% saturated fatty acid, such as 3.5% to 4.5% or 3.5% to 4.0%. Such percentages may be obtained by a fatty acid analysis of the oil as shown in the Examples that follow. In some embodiments, the oil comprises no more than 1% sterols, such as no more than 0.5% or no more than 0.4%. In some embodiments, the oil comprises no more than 0.5% trans fatty acids. In some embodiments, the oil comprises no more than 0.1% tocopherols or no more than 0.15% tocopherols. These are weight percentages based on the total weight of the oil or oil lipids as shown in the Examples that follow.

In some embodiments, the oils comprise phospholipids and also have particular distributions of saturated phospholipids (PLs), the levels of saturated PLs may be similar to that found in higher saturated fat or wild-type oils. In some embodiments, 10-13% of the fatty acids found in the phosphatidyl choline fraction of the oil are saturated fatty acids. In some embodiments, 24-31% of the fatty acids found in the phosphatidyl ethanolamine fraction are saturated. In some embodiments, 17-30% of the fatty acids found in the phostphatidyl inositol fraction are saturated. The percentages of fatty acids in the different phospholipid fractions may be determined as demonstrated in the Examples below.

In some embodiments, the oil has been degummed, refined, bleached, dewaxed, and/or deodorized, for example, by methods described below. In some embodiments, the oil has been emulsified or crystallized, such as to produce a semi-solid state, for example, for preparation of a margarine or shortening.

Brassica Plants with Mutant Alleles and their Preparation

In some embodiments, canola oils may be produced from Brassica plants with particular mutant alleles. Genetic mutations can be introduced within a population of seeds or regenerable plant tissue using one or more mutagenic agents. Suitable mutagenic agents include, for example, ethyl methane sulfonate (EMS), methyl N-nitrosoguanidine (MNNG), ethidium bromide, diepoxybutane, ionizing radiation, x-rays, UV rays and other mutagens known in the art. In some embodiments, a combination of mutagens, such as EMS and MNNG, can be used to induce mutagenesis. The treated population, or a subsequent generation of that population, can be screened for a reduced activity, such as a reduced thioesterase activity, that results from a mutation.

Mutations can be in any portion of a gene, including coding sequence, intron sequence and regulatory elements, that render the resulting gene product non-functional or with reduced activity. Exemplary types of mutations include, for example, insertions or deletions of nucleotides, and transitions or transversions in the wild-type coding sequence. Such mutations can lead to deletion or insertion of amino acids, and conservative or non-conservative amino acid substitutions in the corresponding gene product. In some cases, the mutation is a nonsense mutation, which results in the introduction of a stop codon (TGA, TAA, or TAG) and production of a truncated polypeptide. In some cases, the mutation is a splice site mutation that alters or abolishes the correct splicing of the pre-mRNA sequence, resulting in a protein of different amino acid sequence than the wild type. For example, one or more exons may be skipped during RNA splicing, resulting in a protein lacking the amino acids encoded by the skipped exons. Alternatively, the reading frame may be altered by incorrect splicing, one or more introns may be retained, alternate splice donors or acceptors may be generated, or splicing may be initiated at an alternate position, or alternative polyadenylation signals may be generated. In some cases, more than one mutation or more than one type of mutation is introduced.

Insertions, deletions, or substitutions of amino acids in a coding sequence may, for example, disrupt the conformation of essential alpha-helical or beta-pleated sheet regions of the resulting gene product. Amino acid insertions, deletions, or substitutions also can disrupt binding, alter substrate specificity, or disrupt catalytic sites important for gene product activity. Substitution mutations may be conservative or non-conservative. Non-conservative amino acid substitutions may replace an amino acid of one class with an amino acid of a different class (e.g. replacing a polar or charged amino acid with a non-polar amino acid or an amino acid of opposite charge). Accordingly, examples of non-conservative substitutions include the substitution of a basic amino acid for a non-polar amino acid, or a polar amino acid for an acidic amino acid, or of a basic amino acid for an acidic amino acid or vice versa. Non-conservative substitutions may make a substantial change in the charge or hydrophobicity of the gene product. Non-conservative amino acid substitutions may also make a substantial change in the bulk of the residue side chain even if the general polarity of the amino acid does not change, e.g., substituting an alanine residue for an isoleucine, methionine, or phenylalanine residue. Thus, additional examples of non-conservative amino acid substitutions include replacing a small amino acid such as glycine, alanine, valine, or serine, with a bulky amino acid such as methionine, phenylalanine, tryptophan, or tyrosine, or vice versa. In contrast, conservative substitutions replace amino acids with other amino acids of similar size, polarity, and charge.

In some embodiments, Brassica plants producing canola oils described herein are “non-transgenic,” meaning that they have been obtained without the use of recombinant DNA technology. In contrast, “transgenic” are organisms whose genetic material has been altered using recombinant DNA technology.

Brassica plants producing canola oils described herein may be modified and/or selected to display an herbicide tolerance trait. That trait can be introduced by selection with the herbicide for which tolerance is sought or by use of recombinant DNA techniques. Accordingly, plants described herein may display tolerance to one or more herbicides such as imidazolinone, dicamba, cyclohexanedione, sulfonylurea, glyphosate, flufosinate, phenoxy proprionic acid, L-phosphinothricin, triazine and benzonitrile. In some embodiments, plants may have been modified by transgenic, recombinant DNA technology with regard to their herbicide tolerance trait. Thus, in some embodiments, plants may be free of transgenes aside from those genes conveying their herbicide tolerance traits.

### Exemplary Brassica Gene Mutations

In some embodiments, Brassica plants producing canola oils described herein may have certain gene mutations. In some embodiments, the plants may have reduced thioesterase activity, such as reduced activity of fatty-acyl-ACP thioesterase A2 (FATA2) and/or may have reduced activity of fatty acyl-ACP thioesterase B (FATB). Fatty acyl-ACP thioesterases hydrolyze acyl-ACPs in the chloroplast to release the newly synthesized fatty acid from ACP, effectively removing it from further chain elongation in the plastid. The free fatty acid can then leave the plastid, become bound to CoenzymeA (CoA) and enter the Kennedy pathway in the endoplasmic reticulum (ER) for triacylglycerol (TAG) biosynthesis. Members of the FATA family prefer oleoyl (C18:1) ACP substrates with minor activity towards 18:0 and 16:0-ACPs, while members of the FATB family hydrolyze primarily saturated acyl-ACPs between 8 and 18 carbons in length. See Jones et al., Plant Cell 7:359-371 (1995); Ginalski and Rhchlewski, Nucleic Acids Res. 31:3291-3292 (2003); and Voelker Tin Genetic Engineering (Setlow, JK, Ed.) Vol. 18, pp. 111-133, Plenum Publishing Corp., New York (2003).

Reduced activity, including absence of detectable activity, of FATA2 or FATB can be achieved by modifying an endogenous fatA2 or fatB allele. An endogenous allele can be modified by, for example, mutagenesis, such as with procedures described above, or by using homologous recombination to replace an endogenous plant gene with a variant containing one or more mutations (e.g., produced using site-directed mutagenesis). See, e.g., Townsend et al., Nature 459:442-445 (2009); Tovkach et al., Plant J., 57:747-757 (2009); and Lloyd et al., Proc. Natl. Acad. Sci. USA, 102:2232-2237 (2005). In some embodiments, reduced thioesterase activity can be assessed in plant extracts using assays for fatty acyl-ACP hydrolysis, for example. See, for example, Bonaventure et al., Plant Cell 15:1020-1033 (2003); and Eccleston and Ohlrogge, Plant Cell 10:613-622 (1998).

In some embodiments, a Brassica plant contains a mutant allele at a FATA2 locus, wherein the mutant allele results in the production of a FATA2 polypeptide having reduced thioesterase activity relative to a corresponding wild-type FATA2 polypeptide. For example, the mutant allele can include a nucleic acid that encodes a FATA2 polypeptide having a non-conservative substitution within a helix/4-stranded sheet (4HBT) domain (also referred to as a hot-dog domain) or non-conservative substitution of a residue affecting catalytic activity or substrate specificity. For example, a Brassica plant can contain a mutant allele that includes a nucleic acid encoding a FATA2b polypeptide having a substitution in a region the polypeptide corresponding to residues 242 to 277 of the FATA2 polypeptide (as numbered based on the alignment to the Arabidopsis thaliana FATA2 polypeptide set forth in GenBank Accession No. NP_193041.1, protein; GenBank Accession No. NM_117374, mRNA). See SEQ ID NOs: 12-13. This region of FATA2 is highly conserved in Arabidopsis and Brassica. In addition, many residues in this region are conserved between FATA and FATB, including the aspartic acid at position 259, asparagine at position 263, histidine at position 265, valine at position 266, asparagine at position 268, and tyrosine at position 271 (as numbered based on the alignment to SEQ ID NO:13). The asparagine at position 263 and histidine at position 265 are part of the catalytic triad, and the arginine at position 256 is involved in determining substrate specificity. See also Mayer and Shanklin, BMC Plant Biology 7:1-11 (2007). SEQ ID NO:14 sets forth the predicted amino acid sequence of the Brassica FATA2b polypeptide encoded by exons 2-6, and corresponding to residues 121 to 343 of the A. thaliana sequence set forth in SEQ ID NO:13. For example, the FATA2 polypeptide can have a substitution of a leucine residue for proline at the position corresponding to position 255 of the Arabidopsis FATA2 polypeptide (i.e., position 14 of SEQ ID NO:12 or position 135 of SEQ ID NO:14). The proline in the B. napus sequence corresponding to position 255 in Arabidopsis is conserved among B. napus, B. rapa, B. juncea, Zea mays, Sorghum bicolor, Oryza sativa Indica (rice), Triticum aestivum, Glycine max, Jatropha (tree species), Carthamus tinctorius, Cuphea hookeriana, Iris tectorum, Perilla frutescens, Helianthus annuus, Garcinia mangostana, Picea sitchensis, Physcomitrella patens subsp. Patens, Elaeis guineensis, Vitis vinifera, Elaeis oleifera, Camellia oleifera, Arachis hypogaea, Capsicum annuum, Cuphea hookeriana, Populus trichocarpa, and Diploknema butyracea.

In some embodiments, the mutant allele at a FATA2 locus includes a nucleotide sequence having at least 90% (e.g., at least 91, 92, 93, 94, 95, 96, 97, 98, or 99%) sequence identity to the nucleotide sequence set forth in SEQ ID NO:11 or SEQ ID NO:15. (Determination of sequence identity is described below.) The nucleotide sequences set forth in SEQ ID NOs:11 and 15 are representative nucleotide sequences from the fatA2b gene from B. napus line 15.24.

In some embodiments, a Brassica plant contains a mutant allele at a FATB locus, wherein the mutant allele results in the production of a FATB polypeptide having reduced thioesterase activity relative to a corresponding wild-type FATB polypeptide. In some embodiments, a Brassica plant contains mutant alleles at two or more different FATB loci. In some embodiments, a Brassica plant contains mutant alleles at three different FATB loci or contains mutant alleles at four different FATB loci. Brassica napus contains 6 different FATB isoforms (i.e., different forms of the FATB polypeptide at different loci), which are called isoforms 1-6 herein. SEQ ID NOs:5-10 set forth the nucleotide sequences encoding FATB isoforms 1-6, respectively, of Brassica napus. The nucleotide sequences set forth in SEQ ID NOs:5-10 have 82% to 95% sequence identity as measured by the ClustalW algorithm.

For example, a Brassica plant can have a mutation in a nucleotide sequence encoding FATB isoform 1, isoform 2, isoform 3, isoform 4, isoform 5, or isoform 6. In some embodiments, a plant can have a mutation in a nucleotide sequence encoding isoforms 1 and 2; 1 and 3; 1 and 4; 1 and 5; 1 and 6; 2 and 3; 2 and 4; 2 and 5; 2 and 6; 3 and 4; 3 and 5; 3 and 6; 4 and 5; 4 and 6; 5 and 6; 1, 2, and 3; 1, 2, and 4; 1, 2, and 5; 1, 2, and 6; 2, 3, and 4; 2, 3, and 5; 2, 3, and 6; 3, 4, and 5; 3, 5, and 6; 4, 5, and 6; 1, 2, 3, and 4; 1, 2, 3, and 5; 1, 2, 3, and 6; 1, 2, 4, and 6; 1, 3, 4 and 5; 1, 3, 4, and 6; 1, 4, 5, and 6; 2, 3, 4, and 5; 2, 3, 4 and 6; or 3, 4, 5, and 6. In some embodiments, a Brassica plant can have a mutation in nucleotide sequences encoding FATB isoforms 1, 2, and 3; 1, 2, and 4; 2, 3, and 4; or 1, 2, 3, and 4. In some embodiments, a mutation results in deletion of a 4HBT domain or a portion thereof of a FATB polypeptide. FATB polypeptides typically contain a tandem repeat of the 4HBT domain, where the N-terminal 4HBT domain contains residues affecting substrate specificity (e.g., two conserved methionines, a conserved lysine, a conserved valine, and a conserved serine) and the C-terminal 4HBT domain contains residues affecting catalytic activity (e.g., a catalytic triad of a conserved asparagine, a conserved histidine, and a conserved cysteine) and substrate specificity (e.g., a conserved tryptophan). See Mayer and Shanklin, J. Biol. Chem. 280:3621-3627 (2005). In some embodiments, the mutation results in a non-conservative substitution of a residue in a 4HBT domain or a residue affecting substrate specificity. In some embodiments, the mutation is a splice site mutation. In some embodiment, the mutation is a nonsense mutation in which a premature stop codon (TGA, TAA, or TAG) is introduced, resulting in the production of a truncated polypeptide.

SEQ ID NOs:1-4 set forth the nucleotide sequences encoding isoforms 1-4, respectively, and containing exemplary nonsense mutations that result in truncated FATB polypeptides. SEQ ID NO:1 is the nucleotide sequence of isoform 1 having a mutation at position 154, which changes the codon from CAG to TAG. SEQ ID NO:2 is the nucleotide sequence of isoform 2 having a mutation at position 695, which changes the codon from CAG to TAG. SEQ ID NO:3 is the nucleotide sequence of isoform 3 having a mutation at position 276, which changes the codon from TGG to TGA. SEQ ID NO:4 is the nucleotide sequence of isoform 4 having a mutation at position 336, which changes the codon from TGG to TGA.

Two or more different mutant FATB alleles may be combined in a plant by making a genetic cross between mutant lines. For example, a plant having a mutant allele at a FATB locus encoding isoform 1 can be crossed or mated with a second plant having a mutant allele at a FATB locus encoding isoform 2. Seeds produced from the cross are planted and the resulting plants are selfed in order to obtain progeny seeds. These progeny seeds can be screened in order to identify those seeds carrying both mutant alleles. In some embodiments, progeny are selected over multiple generations (e.g., 2 to 5 generations) to obtain plants having mutant alleles at two different FATB loci. Similarly, a plant having mutant alleles at two or more different FATB isoforms can be crossed with a second plant having mutant alleles at two or more different FATB alleles, and progeny seeds can be screened to identify those seeds carrying mutant alleles at four or more different FATB loci. Again, progeny can be selected for multiple generations to obtain the desired plant.

In some embodiments, plants may comprise a mutant allele at a FATA2 locus as well as mutant alleles at one, two, three, or four different FATB loci. For example, a plant having a mutant allele at a FATA2 locus can be crossed or mated with a second plant having mutant alleles at two or more different FATB loci. Seeds produced from the cross are planted and the resulting plants are selfed in order to obtain progeny seeds. These progeny seeds can be screened in order to identify those seeds carrying mutant FATA2 and FATB alleles. Progeny can be selected over multiple generations (e.g., 2 to 5 generations) to obtain plants having a mutant allele at a FATA2 locus and mutant alleles at two or more different FATB loci. Plants having a mutant allele at a FATA2b locus and mutant alleles at three or four different FATB loci may have a low total saturated fatty acid content that is stable over different growing conditions, i.e., is less subject to variation due to warmer or colder temperatures during the growing season. Due to the differing substrate profiles of the FATB and FATA2 enzymes with respect to C16:0 and C18:0, respectively, plants having mutations in FATA2 and one or more FATB loci may exhibit a substantial reduction in amounts of both C16:0 and C18:0 in seed oil.

In some embodiments, Brassica plants producing canola oils described herein may comprise modified alleles at one or both of the loci termed QTL1 of NO1 and QTL2 of N19 described in PCT publication WO 2015/077661, which is incorporated herein by reference. These modified alleles and methods of identifying them are described in WO 2015/077661. The QTL1 (N1) and QTL2 (N19) modified alleles have been found to correlate to reduced saturated fatty acid content. In some embodiments, plants may have mutations in both the QTL1 and QTL2 loci as well as in FATA2 and/or FATB loci.

In some embodiments, Brassica plants producing canola oils described herein can have reduced fatty acid desaturase activity. For example, in some embodiments, plants can include mutant alleles at loci controlling fatty acid destaturase activity such as fad2 and/or fad3. In some embodiments, a plant may have mutant alleles at one or both of the FATA2 and FATB loci as well as at loci controlling fatty acid desaturatse activity such as fad2 and/or fad3. In some embodiments, a plant may have mutant alleles at one or both of the FATA2 and FATB loci as well as at one or both of QTL1 of NO1 and QTL2 of N19 and at loci controlling fatty acid desaturatse activity such as fad2 and/or fad3.

The fad3 genes encode delta-15 desaturase proteins (also known as FAD3), which are involved in the enzymatic conversion of linoleic acid to a-linolenic acid. There are several isoforms of FAD3, called FAD3A, FAD3B, FAD3C, FAD3D, FAD3E, and FAD3F, encoded by the fad3A, fad3B, fad3C, fad3D, fad3E, fad3F loci, respectively. Sequences of higher plant fad3 genes are disclosed in Yadav et al., Plant Physiol., 103:467-476 (1993), WO 93/11245, and Arondel et al., Science, 258:1353-1355 (1992). Decreased FAD3 activity, including absence of detectable activity, can be inferred from the decreased level of linolenic acid (product) and in some cases, increased level of linoleic acid (the substrate) in the plant compared with a corresponding control plant.

In some embodiments, plants can include a modified allele at a fad3A or fad3B locus, wherein the modified allele results in the production of a FAD3A and/or FAD3B polypeptide having reduced desaturase activity relative to a corresponding wild-type polypeptide. In some embodiments, the parents contain the fad3A and/or fad3B mutation from IMC02 that confer a low linolenic acid phenotype. IMC02 contains a mutation in both the fad3A and fad3B genes and was deposited with the ATCC under Accession No. PTA-6221. In some embodiments, a fad3A mutant may comprise a) a nucleic acid encoding a FAD3A polypeptide having a cysteine substituted for arginine at position 275 and b) a nucleic acid encoding a truncated FAD3A polypeptide. In some embodiments, a fad3B mutant may comprise a) a nucleic acid having a mutation in an exon-intron splice site recognition sequence and b) a nucleic acid encoding a truncated FAD3B polypeptide.

In some embodiments, plants can include a modified allele at a fad3D and/or fad3E locus, wherein the modified allele results in the production of a FAD3D and/or FAD3E polypeptide having reduced desaturase activity relative to a corresponding wild-type polypeptide. A fad3E modified allele can include a nucleic acid encoding a truncated FAD3E polypeptide, a nucleic acid encoding a FAD3E polypeptide having a non-conservative substitution of a residue affecting substrate specificity, or a nucleic acid encoding a FAD3E polypeptide having a non-conservative substitution of a residue affecting catalytic activity. In some embodiments, the fad3E modified allele includes a mutation in a splice donor site. A modified fad3E allele can include a nucleotide sequence having at least 95% sequence identity to the nucleotide sequence set forth in SEQ ID NO:16. A fad3D modified allele can include a nucleic acid encoding a truncated FAD3D polypeptide, a nucleic acid having a deletion of an exon or a portion thereof (e.g., a deletion within exon 1 of the nucleic acid). In some embodiments, a fad3D modified allele includes a nucleotide sequence having at least 95% sequence identity to the nucleic acid sequence set forth in SEQ ID NO:17. In some embodiments, a plant can include fad3E and fad3D modified alleles.

In some embodiments, plants can include a modified allele at a delta-12 fatty acid desaturase (FAD2) locus. The sequences for the wild-type fad2 genes from B. napus (termed the D form and the F form) are disclosed in WO 98/56239. Non-limiting examples of suitable fad2 mutations include the G to A mutation at nucleotide 316 within the fad2D gene, which results in the substitution of a lysine residue for glutamic acid in a HECGH motif. Such a mutation is found within the line IMC129, which has been deposited with the ATCC under Accession No. 40811. Another suitable fad2 mutation can be the T to A mutation at nucleotide 515 of the fad2F gene, which results in the substitution of a histidine residue for leucine in a KYLNNP motif (amino acid 172 of the FAD2F polypeptide). Such a mutation is found within the variety Q508. See U.S. Patent No. 6,342,658. Another example of a fad2 mutation is the G to A mutation at nucleotide 908 of the fad2F gene, which results in the substitution of a glutamic acid for glycine in the DRDYGILNKV amino acid 303 of the FAD2F polypeptide. Such a mutation is found within the line Q4275, which has been deposited with the ATCC under Accession No. 97569. See U.S. Patent No. 6,342,658. Another example of a suitable fad2 mutation can be the C to T mutation at nucleotide 1001 of the fad2F gene (as numbered from the ATG), which results in the substitution of an isoleucine for threonine (amino acid 334 of the FAD2F polypeptide). Such a mutation is found within the high oleic acid line Q7415.

While in some embodiments, Brassica plants may comprise mutations in FAD3 or in FAD2, in other embodiments, the plants do not comprise FAD3 or FAD2 mutations.

### Determining Percent Sequence Identity and Comparing Gene and Protein Sequences

As used herein, the term “sequence identity” refers to the degree of similarity between any given nucleic acid sequence and a target nucleic acid sequence. The degree of similarity is represented as percent sequence identity. Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (Bl2seq) program from the stand-alone version of BLASTZ containing BLASTN version 2.0.14 and BLASTP version 2.0.14. This stand-alone version of BLASTZ can be obtained, for example, from the U.S. government's National Center for Biotechnology Information web site (World Wide Web at “ncbi” dot “nlm” dot “nih” dot “gov”). Instructions explaining how to use the Bl2seq program can be found in the readme file accompanying BLASTZ.

Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. The following command will generate an output file containing a comparison between two sequences: C:\B12seq -i c:\seql.txt -j c:\seq2.txt -p blastn -o c:\output.txt -q -1 -r 2. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive nucleotides from the target sequence presented in alignment with sequence from the identified sequence starting with any matched position and ending with any other matched position. A matched position is any position where an identical nucleotide is presented in both the target and identified sequence. Gaps presented in the target sequence are not counted since gaps are not nucleotides. Likewise, gaps presented in the identified sequence are not counted since target sequence nucleotides are counted, not nucleotides from the identified sequence.

The percent identity over a particular length is determined by counting the number of matched positions over that length and dividing that number by the length followed by multiplying the resulting value by 100. For example, if (i) a 500-base nucleic acid target sequence is compared to a subject nucleic acid sequence, (ii) the Bl2seq program presents 200 bases from the target sequence aligned with a region of the subject sequence where the first and last bases of that 200-base region are matches, and (iii) the number of matches over those 200 aligned bases is 180, then the 500-base nucleic acid target sequence contains a length of 200 and a sequence identity over that length of 90% (i.e., 180, 200×100=90).

It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

### Preparation of Hybrid Brassica Varieties

Hybrid Brassica varieties can be produced by preventing self-pollination of female parent plants (i.e., seed parents), permitting pollen from male parent plants to fertilize such female parent plants, and allowing F₁ hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be cytoplasmic male sterility (CMS), nuclear male sterility, molecular male sterility wherein a transgene inhibits microsporogenesis and/or pollen formation, or be produced by self-incompatibility. Female parent plants containing CMS are particularly useful. CMS can be, for example of the ogu (Ogura), nap, pol, tour, or mur type. See, for example, Pellan-Delourme and Renard, 1987, Proc. 7^{th} Int. Rapeseed Conf., Poznan, Poland, p. 199-203 and Pellan-Delourme and Renard, 1988, Genome 30:234-238, for a description of Ogura type CMS. See, Riungu and McVetty, 2003, Can. J. Plant Sci., 83:261-269 for a description of nap, pol, tour, and mur type CMS.

In embodiments in which the female parent plants are CMS, the male parent plants typically contain a fertility restorer gene to ensure that the F₁ hybrids are fertile. For example, when the female parent contains an Ogura type CMS, a male parent is used that contains a fertility restorer gene that can overcome the Ogura type CMS. Non-limiting examples of such fertility restorer genes include the Kosena type fertility restorer gene (U.S. Pat. No. 5,644,066) and Ogura fertility restorer genes (U.S. Pat. Nos. 6,229,072 and 6,392,127). In other embodiments in which the female parents are CMS, male parents can be used that do not contain a fertility restorer. F₁ hybrids produced from such parents are male sterile. Male sterile hybrid seed can be inter-planted with male fertile seed to provide pollen for seed-set on the resulting male sterile plants.

The methods described herein can be used to form single-cross Brassica F₁ hybrids. In such embodiments, the parent plants can be grown as substantially homogeneous adjoining populations to facilitate natural cross-pollination from the male parent plants to the female parent plants. The F₁ seed formed on the female parent plants is selectively harvested by conventional means. One also can grow the two parent plants in bulk and harvest a blend of F₁ hybrid seed formed on the female parent and seed formed upon the male parent as the result of self-pollination. Alternatively, three-way crosses can be carried out wherein a single-cross F₁ hybrid is used as a female parent and is crossed with a different male parent that satisfies the fatty acid parameters for the female parent of the first cross. Here, assuming a bulk planting, the overall oleic acid content of the vegetable oil may be reduced over that of a single-cross hybrid; however, the seed yield will be further enhanced in view of the good agronomic performance of both parents when making the second cross. As another alternative, double-cross hybrids can be created wherein the F₁ progeny of two different single-crosses are themselves crossed. Self-incompatibility can be used to particular advantage to prevent self-pollination of female parents when forming a double-cross hybrid.

Hybrids described herein may have good agronomic properties and exhibit hybrid vigor, which results in seed yields that exceed that of either parent used in the formation of the F₁ hybrid. For example, yield can be at least 10% (e.g., 10% to 20%, 10% to 15%, 15% to 20%, or 25% to 35%) above that of either one or both parents. In some embodiments, the yield exceeds that of open-pollinated spring canola varieties such as 46A65 (Pioneer) or Q2 (University of Alberta). For example, yield can be at least 10% (e.g., 10% to 15% or 15% to 20%) above that of an open-pollinated variety.

Hybrids described herein may produce seeds having very low levels of glucosinolates (<30 μmol/gram of de-fatted meal at a moisture content of 8.5%). In particular, hybrids can produce seeds having <20 μmol of glucosinolates/gram of de-fatted meal. As such, in some embodiments, hybrids can incorporate mutations that confer low glucosinolate levels. See, for example, U.S. Pat. No. 5,866,762. Glucosinolate levels can be determined in accordance with known techniques, including high performance liquid chromatography (HPLC), as described in ISO 9167-1:1992(E), for quantification of total, intact glucosinolates, and gas-liquid chromatography for quantification of trimethylsilyl (TMS) derivatives of extracted and purified desulfoglucosinolates. Both the HPLC and TMS methods for determining glucosinolate levels analyze de-fatted or oil-free meal.

In some embodiments herein, Brassica plants used to produce canola oils described herein are obtained by crossing parental Brassica breeding lines with particular gene mutations with other Brassica lines that are wild-type at the mutated gene loci and selecting progeny carrying the gene mutations. For example, in some embodiments, Brassica lines with mutations in one or more of QTL1 of N01, QTL2 of N19, FATA2, FATB, FAD3, and FAD2 may be crossed one or more times with lines that are wild-type at those gene loci but that may, for example, have other desirable traits, such as improved yields and/or herbicide tolerance. Markers may be used to select for progeny of such crosses that retain mutations allowing for a desirable triacylglycerol profile on the one hand while retaining mutations for herbicide tolerance or other helpful traits on the other. For example, in some embodiments, markers may be used to select for crosses retaining mutations in the one or more of QTL1 of N01, QTL2 of N19, FATA2, FATB, FAD3, and FAD2. In some embodiments, markers may be used to select for crosses retaining mutations in the one or more of QTL1 of N01, QTL2 of N19, FATA2, FATB, FAD3, and FAD2 as well as mutations conferring herbicide tolerance.

In some embodiments, lines with mutations in one or more of QTL1 of N01, QTL2 of N19, FATA2, FATB, FAD3, and FAD2 may be crossed with one or more high-yielding Brassica lines, including high-yielding Brassica lines that also possess herbicide tolerance, such as the parental lines 03LC.034 and 07RF543.001of Cargill VICTORY® hybrid V12 canola lines. In some embodiments, the initial crosses may be back-crossed one or more times with the high-yielding Brassica line and progeny selected that retain mutations in one or more of QTL1 of N01, QTL2 of N19, FATA2, FATB, FAD3, and FAD2, and optionally that also retain mutations that give rise to or correlate with herbicide tolerance and high yield.

In some embodiments, the resulting hybrid plant breeding lines have a yield that is at least 10%, such as 10% to 15%, 10% to 20%, 10% to 25%, 10% to 35%, 15% to 20%, 15% to 25%, 20% to 25%, or 25% to 35% above that of either one or both parental breeding lines. In some embodiments, the hybrid plant breeding lines have a yield that is at least 10%, such as 10% to 15%, 10% to 20%, 10% to 25%, 10% to 35%, 15% to 20%, 15% to 25%, 20% to 25%, or 25% to 35% above that of the highest yielding parental line. In some embodiments, the yield exceeds that of open-pollinated spring canola varieties such as 46A65 (Pioneer) or Q2 (University of Alberta). For example, in such embodiments, yield can be at least 10%, such as 10% to 15%, 10% to 20%, or 15% to 20% above that of an open-pollinated variety such as 46A65 (Pioneer) or Q2 (University of Alberta).

In the context of this application, “yield” may be measured in units of grain weight per area, for example, in Kg per square meter or Kg per hectare or bushels per acre.

In the context of this application, comparisons of yields of two canola lines or varieties may be conducted such that parameters that may affect growth, such as sunlight, temperature, soil conditions, moisture, fertilizer and pest control, weed control, and seeding rate and depth are controlled. For example, to compare yields of different plant lines, each line or variety could be planted, grown, and harvested within the same field, greenhouse, or growth chamber using standard randomization and replication methodology (i.e. randomized complete block design) so each genotype may experience the range of uncontrollable variation existing within each type of growth condition. To determine yield, at seed maturity the plots may be swathed, and the swath allowed to dry, after which the swath may be harvested with a combine and grain weight determined.

In some embodiments, plants may be hybrids between lines with mutations in one or more of QTL1 of N01, QTL2 of N19, FATA2, FATB, FADS, and FAD2 and one or more high-yielding Brassica lines, including high-yielding Brassica lines that also possess herbicide tolerance, such as the parental lines 03LC.034 and 07RF543.001of Cargill VICTORY® hybrid V12 canola lines. For example, such hybrid lines and their progeny, including further back-crosses, may have a yield that varies from that of the high-yielding Brassica parental lines by no more than 20%, by no more than 15%, by no more than 10%, or by no more than 5%.

### Preparation of Oils

Oils may be prepared, for example, from Brassica seeds. For example, seeds may be flaked and heat-conditioned and then passed through a screw-press or similar device to release oils. Crude oil produced from the pressing operation may be clarified by passing the crude oil through a settling tank with a slotted wire drainage top to remove particulates. The oil can then be passed through a plate and frame filter to remove the remaining fine particulates, resulting in a clarified oil. The press cake can also be extracted with commercial n-Hexane to extract additional oil. The canola oil recovered from the extraction process can be combined with the clarified oil from the screw pressing operation, resulting in a blended crude oil.

Oils may also be treated to remove phosphatides, metal salts, gums, and free fatty acids. For example, a degumming procedure may be used to remove phosphatides co-extracted with the oil. Phosphatides may separate from the oil upon storage, forming a sludge, and thus, it may be desirable to remove them. Possible degumming procedures include using water to precipitate phosphatides, using a water/acid mixture, or using a mixture of acid and aqueous sodium hydroxide to saponify phosphatides and other impurities such as free fatty acids.

The oil, such as a degummed oil, may also be refined, such as in an alkali refining process. For example, in an alkali refining process, oil may be contacted with, for instance, 0.05-0.1% phosphoric acid and intensely mixed and then with about 12% aqueous sodium hydroxide, which may neutralize free fatty acids as well as any remaining phosphoric acid and precipitate remaining phosphatides. An aqueous soap phase is thus created including phosphatides, neutralized free fatty acids, and metal salts, which may then be removed by centrifugation.

The oil may also be bleached. For example, bleaching may be used to remove chlorophyll compounds that may oxidize the oil or give it an undesirable greenish color. In some cases, bleaching and refining may be performed concurrently in a process of physical refining in which phosphoric acid and alkali treatments are combined with exposure to bleaching clay to adsorb chlorophylls.

In some cases, oils may also be dewaxed, i.e., have waxy substances removed. As waxy substances tend to precipitate at room temperature while other oil components remain liquid, this process may help the oil to remain clear upon storage.

Oils may also be deodorized, for example, to remove substances from seeds that impart unwanted odors and tastes to the oil. For example, the oil may be steam distilled to remove relatively volatile compounds.

For some uses, oils may also be emulsified or crystallized into a semi-solid form, such as to create margarine or shortening.

Oils described herein may in some embodiments have increased oxidative stability compared to wild-type plants. Oxidative stability can be measured using, for example, an Oxidative Stability Index Instrument (e.g., from Omnion, Inc., Rockland, Mass.) according to AOCS Official Method Cd 12b-92 (revised 1993). Oxidative stability is often expressed in terms of “AOM” hours.

### Food Compositions and Uses of Canola Oils

Uses of oils to prepare food compositions and associated food compositions are also provided herein. For example, the instant oils can be used to replace or reduce the amount of saturated fatty acids and hydrogenated oils (e.g., partially hydrogenated oils) in various food products such that the levels of saturated fatty acids and trans fatty acids are reduced in the food products. In particular, the oils can be used to replace or reduce the amount of saturated fats and partially hydrogenated oils in processed or packaged food products, including bakery products such as cookies, muffins, doughnuts, pastries (e.g., toaster pastries), pie fillings, pie crusts, pizza crusts, frostings, breads, biscuits, and cakes, breakfast cereals, breakfast bars, puddings, and crackers.

For example, an oil described herein can be used to produce sandwich cookies that contain reduced saturated fatty acids and no or reduced levels of partially hydrogenated oils in the cookie and/or creme filling. Such a cookie composition can include, for example, in addition to canola oil, flour, sweetener (e.g., sugar, molasses, honey, high fructose corn syrup, artificial sweetener such as sucralose, saccharine, aspartame, or acesulfame potassium, and combinations thereof), eggs, salt, flavorants (e.g., chocolate, vanilla, or lemon), a leavening agent (e.g., sodium bicarbonate or other baking acid such as monocalcium phosphate monohydrate, sodium aluminum sulfate, sodium acid pyrophosphate, sodium aluminum phosphate, dicalcium phosphate, glucano-deltalactone, or potassium hydrogen tartrate, or combinations thereof), and optionally, an emulsifier (e.g., mono- and diglycerides of fatty acids, propylene glycol mono- and di-esters of fatty acids, glycerol-lactose esters of fatty acids, ethoxylated or succinylated mono- and diglycerides, lecithin, diacetyl tartaric acid esters or mono- and diglycerides, sucrose esters of glycerol, and combinations thereof). A creme filling composition can include, in addition to canola oil, sweetener (e.g., powdered sugar, granulated sugar, honey, high fructose corn syrup, artificial sweetener, or combinations thereof), flavorant (e.g., vanilla, chocolate, or lemon), salt, and, optionally, emulsifier.

Canola oils described herein also may be useful for frying applications due to the polyunsaturated content, which, in some embodiments, is low enough that it may have improved oxidative stability for frying yet high enough to impart the desired fried flavor to the food being fried. For example, canola oils can be used to produce fried foods such as snack chips (e.g., corn or potato chips), French fries, or other quick serve foods.

Oils described herein also can be used to formulate spray coatings for food products (e.g., cereals or snacks such as crackers). In some embodiments, the spray coating can include other vegetable oils such as sunflower, cottonseed, corn, or soybean oils. A spray coating also can include an antioxidant and/or a seasoning.

Oils described herein also can be use in the manufacturing of dressings, mayonnaises, and sauces to provide a reduction in the total saturated fat content of the product. The low saturate oil can be used as a base oil for creating structured fat solutions such as microwave popcorn solid fats or canola butter formulations.

The invention will be further described in the following examples, which do not limit the scope of the invention described in the claims.

### EXAMPLES

### Example 1

### Preparation and Genotypes of Brassica Plants

Inbred Brassica napus lines used for seed production and subsequent oil analysis were developed using a marker-assisted breeding program. Mutant alleles of breeding lines Salomon-05 (ATCC accession number PTA-11453) and mIMC201 were introgressed into high-yielding low linolenic (C18:3) breeding lines 03LC.034 and 07RF543.001 03LC.034 and 07RF543.001 are the parents of the V12-1, a registered hybrid variety in Canada. The development and characterization of the Salomon-05 breeding line mutant allele is described in Example 8 of PCT publication WO 2011/075716. The development and characterization of the mutant alleles in the fatty acyl-ACP thioesterase B (FATB) isoforms of mIMC201 are described in Examples 4, 5 and 6 of WO 2011/075716.

Breeding of low sat mutant alleles into 03LC.034 and 07RF543.001 was performed to understand the impact of these alleles in genetic backgrounds other than those in which they were discovered (i.e. Salmon and mIMC201). It has been demonstrated that the phenotypic effect of an allele can vary as it is transferred into new genetic backgrounds carrying alternative alleles at loci throughout the genome (L. Lecomte et al., Theoretical and Applied Genetics, Vol. 109, No. 3, 658-668 (2004).) Introgression was also initiated to understand the impacts of these low sat alleles and resulting low saturate phenotypes on agronomic performance. Previous research has shown that reductions in total saturated fat may result in poor plant performance (Bonaventure et al., The Plant Cell. Vol. 15, No. 4, 1020-1033 (2003); Cardinal et al., Crop Science. Vol. 47 No. 1, 304-310 (2008).)

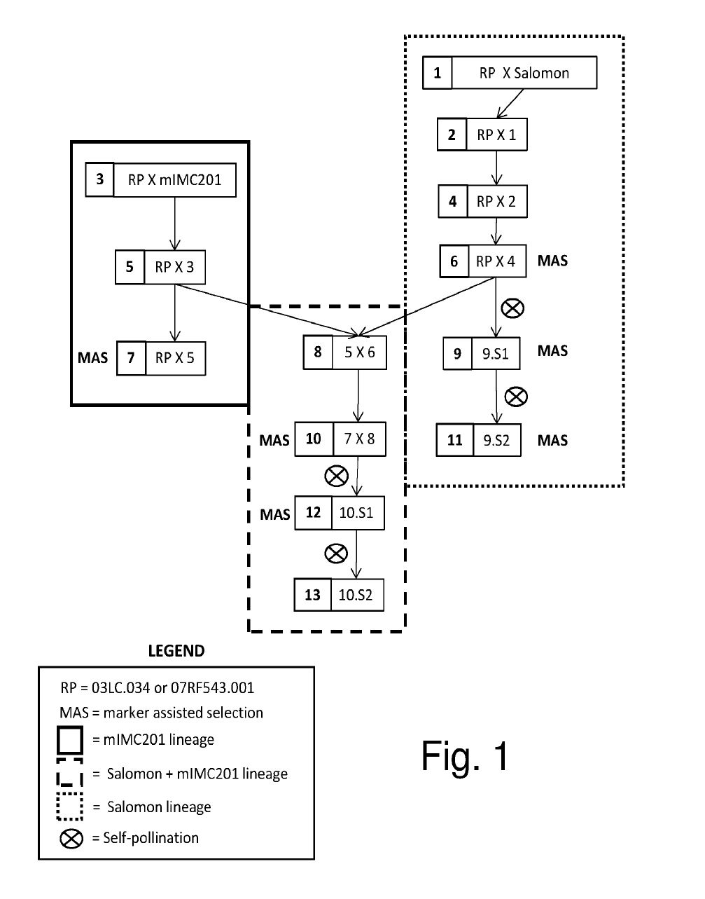

Molecular markers associated with two quantitative trait loci (QTL) on chromosomes N01 and N19 (hereafter referred to as ‘QTL alleles’), previously described in PCT publication WO 2015/077661, the FATA2 mutation described in WO 2011/075716 and the FATB mutant alleles described in WO 2011/075716 were used to assist a backcross breeding program to introgress these loci into 03LC.034 and 07RF543.001 (hereafter referred to as the recurrent parents, RP). Salomon-05 and mIMC201 were crossed with the recurrent parents. (See FIG. 1.) The resulting F1s were backcrossed to the recurrent parent two to three times (generations 1-7 in FIG. 1). The Salomon backcrossing lineage was self-pollinated (generation 9, Figurel) followed by marker-assisted selection to create BC3S2 seeds homozygous for the QTL alleles and the FATA2 mutant allele (generation 11, FIG. 1). To merge mutant alleles, a cross was made between the Salomon lineage and the mIMC201 lineage (generation 8, FIG. 1). After another generation of backcrossing (generation 10, FIG. 1), plants were self-pollinated for one generation (generation 12, FIG. 1) followed by marker assisted selection to create BC3S2 seeds homozygous for the QTL alleles, the FATA2 mutant allele and various combinations of FATB mutant alleles (generation 13, FIG. 1). Ultimately, BC3S2 selections described in Table 2 were used for planting in chambers to create the seeds used in the analyses of Examples 3-5 below.

The plants listed in Table 2 were homozygous for each of the mutant alleles listed, with the exception of the H07/L07 plants, which were heterozygous for FATB2, but homozygous for the other listed mutant alleles.

TABLE 2
Experimental samples, pedigrees, and genotypes

| Sample | | |
|---|---|---|
| Name | Mutant Alleles | Pedigree |
| H11/L11 | | RO11*S × GA106-45 |
| H20/L20 | | Wings06 × 03RF08.09 |
| H01/L01 | N01, N19, FATA2 | 03LC.034*3/Salomon |
| H05/L05 | N01, N19, FATA2, | 03LC.034*3/Salomon// |
| | FATB1/4 | 03LC.034*2/mIMC201 |
| H07/L07 | N01, N19, FATA2, | 03LC.034*3/Salomon// |
| | FATb2B2/3/4 | 03LC.034*2/mIMC201 |
| | (heterozygous in | |
| | FATB2) | |
| H10/L10 | N01, N19, FATA2, | 03LC.034*3/Salomon// |
| | FATB1/2/3/4 | 03LC.034*2/mIMC201 |
| H12/L12 | N01, N19, FATA2 | 07RF543.001*3/Salomon |
| H17/L17 | N01, N19, FATA2, | 07RF543.001*3/Salomon// |
| | FATB1/4 | 07RF543.001*2/IMC201Mutant |
| H18/L18 | N01, N19, FATA2, | 07RF543.001*3/Salomon// |
| | FATB3/4 | 07RF543.001*2/IMC201Mutant |
| H19/L19 | N01, N19, FATA2, | 07RF543.001*3/Salomon// |
| | FATEB1/3/4 | 07RF543.001*2/IMC201Mutant |

### Example 2

### Plant Growing Conditions and Oil Sample Preparation

Plants from Example 1, Table 2, were grown in either high (H) or low (L) temperature conditions, and seeds were collected for analysis. In the H (high) temperature chambers the plants were grown at day temp of 20° C. and night temp of 17° C.; for the L (low) temperature chambers the day temps were 15° C. and the night temps were 12° C.

Specifically, seeds were planted in Premier Pro-Mix BX potting soil (Premier Horticulture, Quebec, Canada) in four inch plastic pots. Planted seeds were watered and germinated at 20° C. day (16 hours light) and 17° C. night (8 hours dark) in Conviron ATC60 controlled-environment growth chambers (Controlled Environments, Winnipeg, MB). Each genotype was randomized and replicated 10 times in each of two separate growth chambers. At the onset of flowering, one chamber was reduced to a diurnal temperature cycle of 15° C. day temperature and 12° C. night temperature (the low temperature treatment, L) while the other remained at the original planting temperature of 20° C. day and 17° C. night. Plants were watered five times per week and fertilized bi-weekly using a 20:20:20 (NPK) liquid fertilizer at a rate of 150 ppm. Plants were bagged individually to ensure self pollination and genetic purity of the seed. Seeds from were harvested at physiological maturity. All plants were analyzed using PCR based assays to confirm the presence of the mutant alleles.

To prepare samples for analyses of fatty acid content, triacylglycerol profile, content of various lipid classes, and phospholipid profile, about 7-20 g of seeds was placed in a press chamber (Carver laboratory press model C), and pressed for 5 minutes. Pressed oil was transferred from the sample collection container to an amber vial. The pressed seeds (meal) were immediately transferred to a 250 Erlenmeyer flask and mixed with n-hexane at a ratio of 1:2.5 pressed seeds/n-hexane. Samples were capped and placed in a shaker (Triad Scientific Multi-Wrist Shaker) set to “slow” for 1 hour at room temperature (e.g., 23° C.). After 1 hour, the shaken meal mixture was filtered through Whatman#4 filter paper. Filtrates from duplicate flasks were combined and the hexane was evaporated using a rotary evaporator set at 60° C. and 100 rpm (Rotovapor 215, Buchi). Pressed and hexane extracted oils were then combined, yielding about 3-6 g of oil for each seed sample. Approximately 1 g of each oil sample was used for further analysis procedures.

### Example 3

### Fatty Acid and Triacylglycerol Profile Analysis

The overall lipid and fatty acid composition of each sample is shown in tables 3a and 3b below. Control samples are shaded in grey. In these tables, TAG, DAG, and MAG stand for tri-, di-, and monoglycerides, respectively, FFA stands for free fatty acids, “toco” stands for tocopherol, and “T” indicates a trans fatty acid. “Totalsats” is the total saturated fatty acids while “totaltrans” is the total trans fatty acids.

TABLE 3a
Fatty acid composition H samples

| Sample ID | H01 | H05 | H07 | H10 | H11 | H12 | H17 | H18 | H19 | H20 |
|---|---|---|---|---|---|---|---|---|---|---|
| % TAG (rep1) | 96.4245 | 94.9900 | 96.4820 | 96.1060 | 96.8838 | 97.1521 | 96.6738 | 95.5067 | 96.7890 | 97.4410 |
| % TAG (rep2) | 96.7721 | 95.4441 | 96.6630 | 96.2544 | 97.1549 | 96.9913 | 96.8805 | 95.8606 | 96.6635 | 97.0234 |
| % TAG (avg) | 96.5983 | 95.2171 | 96.5725 | 96.1802 | 97.0194 | 97.0717 | 96.7772 | 95.6837 | 96.7263 | 97.2322 |
| % DAG (rep1) | 2.7784 | 4.1025 | 2.9798 | 3.2001 | 2.4925 | 2.2375 | 2.4020 | 2.9526 | 2.5725 | 2.1046 |
| % DAG (rep2) | 2.7346 | 3.2705 | 2.3840 | 3.0752 | 2.2522 | 2.2295 | 2.3395 | 2.6812 | 2.5843 | 2.2696 |
| % DAG (avg) | 2.7565 | 3.6865 | 2.6819 | 3.1377 | 2.3724 | 2.2335 | 2.3708 | 2.8169 | 2.5784 | 2.1871 |
| % MAG + FFA 1 | 0.6682 | 0.4777 | 0.3633 | 0.4311 | 0.5703 | 0.5569 | 0.6563 | 0.6388 | 0.5945 | 0.4052 |
| % MAG + FFA 2 | 0.4797 | 0.6360 | 0.4433 | 0.5502 | 0.5531 | 0.5953 | 0.7017 | 0.7147 | 0.6801 | 0.6108 |
| % MAG + FFA | 0.5740 | 0.5569 | 0.4033 | 0.4907 | 0.5617 | 0.5761 | 0.6790 | 0.6768 | 0.6373 | 0.5080 |
| α Toco (ppm) | 177.522 | 341.944 | 295.987 | 293.732 | 267.121 | 243.312 | 366.872 | 370.353 | 367.244 | 305.99 |
| γ Toco (ppm) | 439.495 | 462.975 | 461.387 | 430.825 | 475.134 | 335.702 | 487.872 | 431.074 | 465.937 | 565.75 |
| δ Toco (ppm) | 5.102 | 5.872 | 6.251 | 6.55 | 7.607 | 2.006 | 7.714 | 6.283 | 4.537 | 7.502 |
| Total TOCO | 622.119 | 810.791 | 763.625 | 731.107 | 749.862 | 581.02 | 862.458 | 807.71 | 837.718 | 879.242 |
| C14:0 | 0.0091 | 0.0081 | 0.0084 | 0.0075 | 0.0271 | 0.0101 | 0.0094 | 0.0072 | 0.0086 | 0.0248 |
| C14:1T | 0.0132 | 0.0150 | 0.0158 | 0.0158 | 0.0214 | 0.0164 | 0.0189 | 0.0000 | 0.0160 | 0.0000 |
| C16:0 | 3.2991 | 2.5013 | 2.5934 | 2.7350 | 4.7051 | 3.0625 | 2.8501 | 2.7833 | 2.8248 | 4.4949 |
| C16:1T | 0.0377 | 0.0368 | 0.0349 | 0.0433 | 0.0398 | 0.0384 | 0.0435 | 0.0461 | 0.0439 | 0.0322 |
| C16:1 | 0.0538 | 0.0714 | 0.0709 | 0.0735 | 0.1608 | 0.0763 | 0.0799 | 0.0746 | 0.0731 | 0.2528 |
| C18:0 | 1.1475 | 1.0773 | 0.9950 | 1.3968 | 2.1167 | 0.9202 | 0.8962 | 0.8742 | 0.8045 | 1.4354 |
| C18:1T | 0.0000 | 0.0000 | 0.0000 | 0.0000 | 0.0000 | 0.0000 | 0.0000 | 0.0000 | 0.0000 | 0.0000 |
| C18:1T | 0.0000 | 0.0000 | 0.0000 | 0.0000 | 0.0000 | 0.0000 | 0.0000 | 0.0000 | 0.0000 | 0.0000 |
| C18:1T | 0.0000 | 0.0000 | 0.0000 | 0.0000 | 0.0000 | 0.0000 | 0.0000 | 0.0000 | 0.0000 | 0.0000 |
| C18:1 | 64.2930 | 62.6314 | 63.6882 | 64.2338 | 66.4184 | 66.6768 | 73.0172 | 72.5509 | 67.3231 | 72.0972 |
| C18:2TT | 0.0000 | 0.0000 | 0.0000 | 0.0000 | 0.0000 | 0.0000 | 0.0000 | 0.0000 | 0.0000 | 0.0000 |
| C18:2 | 24.0428 | 26.7067 | 25.4316 | 24.5613 | 20.5098 | 22.8115 | 16.6094 | 16.9548 | 22.4734 | 15.8115 |
| C20:0 | 0.4454 | 0.2854 | 0.3545 | 0.3803 | 0.5332 | 0.4091 | 0.4232 | 0.3976 | 0.4071 | 0.6167 |
| C20:1T | 0.0000 | 0.0000 | 0.0000 | 0.0000 | 0.0000 | 0.0000 | 0.0000 | 0.0000 | 0.0000 | 0.0000 |
| C20:1 | 1.4024 | 1.0999 | 1.3713 | 1.1431 | 0.7411 | 1.6624 | 1.6237 | 1.6599 | 1.6453 | 1.1136 |
| C18:3 | 4.1082 | 4.7219 | 4.4898 | 4.4731 | 3.7066 | 2.9456 | 3.0524 | 3.2358 | 3.0057 | 2.7608 |
| C20:2 | 0.1511 | 0.1510 | 0.1548 | 0.1442 | 0.0660 | 0.1841 | 0.1537 | 0.1662 | 0.1705 | 0.0617 |
| C22:0 | 0.2543 | 0.1370 | 0.2156 | 0.1512 | 0.2038 | 0.3232 | 0.3324 | 0.3339 | 0.3249 | 0.4394 |
| C22:1 | 0.0000 | 0.0000 | 0.0000 | 0.0000 | 0.0000 | 0.0329 | 0.0371 | 0.0477 | 0.0314 | 0.0000 |
| C22:1T | 0.0000 | 0.0000 | 0.0000 | 0.0000 | 0.0000 | 0.0000 | 0.0000 | 0.0000 | 0.0000 | 0.0000 |
| C24:0 | 0.1948 | 0.0715 | 0.1631 | 0.1271 | 0.1316 | 0.1967 | 0.2277 | 0.2334 | 0.2132 | 0.2430 |
| C24:1 | 0.1446 | 0.0944 | 0.1502 | 0.1093 | 0.1181 | 0.1471 | 0.1576 | 0.1549 | 0.1547 | 0.1378 |
| TotalSats | 5.3501 | 4.0806 | 4.3302 | 4.7978 | 7.7175 | 4.9218 | 4.7390 | 4.6295 | 4.5831 | 7.2542 |
| TotalTrans | 0.4463 | 0.4351 | 0.3078 | 0.4639 | 0.5515 | 0.5322 | 0.5216 | 0.5019 | 0.5305 | 0.4820 |

TABLE 3b
Fatty acid composition of L samples

| Sample ID | L01 | L05 | L07 | L10 | L11 | L12 | L17 | L18 | L19 | L20 |
|---|---|---|---|---|---|---|---|---|---|---|
| % TAG (rep1) | 96.0729 | 96.2157 | 96.1173 | 96.5527 | 96.6115 | 96.4105 | 96.6749 | 96.6872 | 96.6119 | 96.9226 |
| % TAG (rep2) | 96.2352 | 96.3500 | 96.0126 | 96.5092 | 96.4669 | 96.5013 | 96.5311 | 96.8345 | 96.4980 | 96.8889 |
| % TAG (avg) | 96.1541 | 96.2829 | 96.0650 | 96.5310 | 96.5392 | 96.4559 | 96.6030 | 96.7609 | 96.5550 | 96.9058 |
| % DAG (rep1) | 3.2853 | 3.0094 | 2.9796 | 2.9121 | 2.8706 | 2.7240 | 2.7187 | 2.7112 | 2.7700 | 2.5281 |
| % DAG (rep2) | 3.0335 | 2.8381 | 3.2076 | 2.9356 | 2.8773 | 2.7254 | 2.7092 | 2.6151 | 2.8122 | 2.4805 |
| % DAG (avg) | 3.1594 | 2.9238 | 3.0936 | 2.9239 | 2.8740 | 2.7247 | 2.7140 | 2.6632 | 2.7911 | 2.5043 |
| % MAG + FFA 1 | 0.4724 | 0.6224 | 0.7249 | 0.4104 | 0.4682 | 0.7226 | 0.5197 | 0.5080 | 0.5892 | 0.5338 |
| % MAG + FFA 2 | 0.6553 | 0.5954 | 0.5588 | 0.5334 | 0.4323 | 0.7505 | 0.5187 | 0.4846 | 0.5792 | 0.5414 |
| % MAG + FFA | 0.5639 | 0.6089 | 0.6419 | 0.4719 | 0.4503 | 0.7366 | 0.5192 | 0.4963 | 0.5842 | 0.5376 |
| α Toco (ppm) | 201.647 | 254.725 | 262.078 | 228.293 | 233.956 | 210.757 | 298.27 | 282.108 | 288.186 | 223.383 |
| γ Toco (ppm) | 413.431 | 379.152 | 413.319 | 368.961 | 390.605 | 314.462 | 400.991 | 413.897 | 374.325 | 468.962 |
| δ Toco (ppm) | 4.562 | 4.615 | 5.56 | 4.747 | 6.506 | 0.924 | 3.49 | 3.049 | 1.929 | 4.938 |
| Total TOCO | 619.64 | 638.492 | 680.957 | 602.001 | 631.067 | 526.143 | 702.751 | 699.054 | 664.44 | 697.283 |
| C14:0 | 0.0074 | 0.0058 | 0.0050 | 0.0065 | 0.0228 | 0.0088 | 0.0056 | 0.0059 | 0.0040 | 0.0199 |
| C14:1T | 0.0126 | 0.0185 | 0.0155 | 0.0172 | 0.0233 | 0.0173 | 0.0000 | 0.0000 | 0.0000 | 0.0216 |
| C16:0 | 3.2685 | 2.2408 | 2.4961 | 2.6046 | 4.6296 | 2.8770 | 2.4429 | 2.4445 | 2.3241 | 4.5310 |
| C16:1T | 0.0334 | 0.0309 | 0.0350 | 0.0341 | 0.0332 | 0.0334 | 0.0371 | 0.0323 | 0.0376 | 0.0276 |
| C16:1 | 0.0690 | 0.0937 | 0.0906 | 0.0829 | 0.2101 | 0.0808 | 0.0853 | 0.0831 | 0.0921 | 0.2661 |
| C18:0 | 1.1961 | 1.4396 | 0.9591 | 1.3654 | 2.3006 | 0.8171 | 0.6753 | 0.7053 | 0.6926 | 1.3249 |
| C18:1T | 0.0000 | 0.0000 | 0.0000 | 0.0000 | 0.0144 | 0.0000 | 0.0000 | 0.0000 | 0.0000 | 0.0000 |
| C18:1T | 0.0000 | 0.0000 | 0.0000 | 0.0000 | 0.0000 | 0.0000 | 0.0000 | 0.0000 | 0.0000 | 0.0000 |
| C18:1T | 0.0000 | 0.0000 | 0.0000 | 0.0000 | 0.0000 | 0.0000 | 0.0000 | 0.0000 | 0.0000 | 0.0000 |
| C18:1 | 60.5600 | 63.1846 | 60.2879 | 61.4231 | 64.9173 | 63.1323 | 67.3870 | 67.5229 | 62.2449 | 68.8185 |
| C18:2TT | 0.0000 | 0.0000 | 0.0000 | 0.0000 | 0.0000 | 0.0192 | 0.0000 | 0.0000 | 0.0000 | 0.0000 |
| C18:2 | 27.6210 | 26.1404 | 28.2360 | 27.4428 | 21.4189 | 26.2088 | 21.9319 | 21.7115 | 27.3341 | 18.6601 |
| C20:0 | 0.4905 | 0.3761 | 0.3759 | 0.3352 | 0.5461 | 0.3599 | 0.3372 | 0.3589 | 0.3298 | 0.5750 |
| C20:1T | 0.0000 | 0.0000 | 0.0000 | 0.0000 | 0.0000 | 0.0000 | 0.0000 | 0.0000 | 0.0000 | 0.0000 |
| C20:1 | 1.3821 | 1.0495 | 1.3790 | 1.0205 | 0.7038 | 1.6072 | 1.6516 | 1.7357 | 1.6128 | 1.1297 |
| C18:3 | 4.0618 | 4.5650 | 4.8627 | 4.7512 | 4.1970 | 3.7537 | 4.1140 | 4.0663 | 4.0169 | 3.3583 |
| C20:2 | 0.1638 | 0.1378 | 0.1879 | 0.1507 | 0.0604 | 0.1890 | 0.1795 | 0.1780 | 0.2001 | 0.0656 |
| C22:0 | 0.3059 | 0.1556 | 0.2539 | 0.1480 | 0.2070 | 0.3145 | 0.3069 | 0.3287 | 0.3129 | 0.4583 |
| C22:1 | 0.0000 | 0.0000 | 0.0000 | 0.0000 | 0.0000 | 0.0000 | 0.0000 | 0.0000 | 0.0000 | 0.0000 |
| C22:1T | 0.0000 | 0.0000 | 0.0000 | 0.0000 | 0.0000 | 0.0268 | 0.0354 | 0.0391 | 0.0399 | 0.0000 |
| C24:0 | 0.2137 | 0.0559 | 0.1494 | 0.1011 | 0.1182 | 0.1443 | 0.1457 | 0.1551 | 0.1417 | 0.1646 |
| C24:1 | 0.2285 | 0.1264 | 0.2351 | 0.1377 | 0.1496 | 0.1913 | 0.2167 | 0.2355 | 0.2132 | 0.1977 |
| TotalSats | 5.4821 | 4.2738 | 4.2394 | 4.5607 | 7.8242 | 4.5215 | 3.9135 | 3.9984 | 3.8052 | 7.0737 |
| TotalTrans | 0.4249 | 0.4208 | 0.4713 | 0.4221 | 0.5110 | 0.3115 | 0.5128 | 0.4626 | 0.4600 | 0.4233 |

Table 4 below shows the weight percent of triacylglycerols (TAGs) comprising one, two, or three saturated fatty acids (columns marked “total monosats,” “total disats,” and “total trisats,” and the amount of TAGs comprising three unsaturated fatty acids “total triunsats” normalized to a total TAG weight percent of 100%. Tables 5a and 5b below show the weight percent of each detectable TAG species, with the fatty acid abbreviations present in that species given in Table 1 above. Lanes showing oils produced from genotypically wild-type Brassica strains are shown in grey in both tables below. Note that each listed TAG species is a sum of its individual isomers. In other words, the method does not distinguish TAG species comprising the same group of three fatty acids but where the different fatty acids are found at different positions on the glycerol (i.e. the SN₁, SN₂ and SN₃ positions). Thus, for example, the abbreviation “POO” in Table 5b below includes all TAGs with one P and two O fatty acids, regardless of which position on the glycerol those fatty acids are each located.

Triacylglycerols in oil samples were detected by reversed phase ultra-performance liquid chromatography (UPLC®; Waters Corporation) coupled to an evaporative light scattering detector (ELSD) system (Waters Corporation), using ZORBAX® Eclipse Plus C18 RRHD 2.1×150 mm, 1.8 micron column (P/N 959759-902, Agilent) and ZORBAX® Eclipse Plus C18 RRHD 2.1×100 mm, 1.8 micron column (P/N 959758-902, Agilent) in series. The column temperature was maintained at 55° C. with a mobile phase of A: methanol and B: 50:50 acetone : ethyl acetate, and a flow rate of 0.5 mL/minute.

Purified TAGs LaLaLa, MMM, LLL, POP, SPS, SSS, and OOO and the DAG 1,3-dipalmitin (PP) were used to develop calibrations curves (Nu-Chek Prep, Inc., Elysian, Minn., USA, product nos. T-130, T-140, T-250, T160, T225, Indofine Chemical Co., Inc., Hillsborough, N.J., USA, product nos. 34-1611 and 34-1810, and Nu-Chek Prep no. D-152, respectively). C39 TAG (Nu-Chek Prep no. T-135) was used as an internal standard (IS).

The amount of each TAG species was determined based upon the known retention time for that species. Quantification was based upon a multi-level calibration curve generating an internal standard response factor using an appropriate TAG as an external standard and a TAG not present in the oil as an IS.

TABLE 4
TAG Analysis

| Sample | Total Monosats | Total Disats | Total Trisats | Total Triunsats |
|---|---|---|---|---|
| H01 | 15.6 | ND | ND | 82.4 |
| H05 | 12.1 | ND | ND | 85.8 |
| H07 | 13.4 | ND | ND | 84.5 |
| H10 | 14.2 | ND | ND | 83.9 |
| H11 | 21.6 | ND | ND | 76.9 |
| H12 | 15.3 | ND | ND | 83.4 |
| H17 | 14.6 | ND | ND | 84.4 |
| H18 | 14.2 | ND | ND | 85.0 |
| H19 | 13.5 | ND | ND | 85.0 |
| H20 | 20.0 | ND | ND | 79.1 |
| L05 | 12.5 | ND | ND | 85.9 |
| L10 | 12.7 | ND | ND | 85.2 |
| L07 | 13.1 | ND | ND | 84.7 |
| L17 | 12.3 | ND | ND | 86.1 |
| L18 | 13.9 | ND | ND | 84.8 |
| L01 | 15.1 | ND | ND | 83.1 |
| L11 | 20.5 | ND | ND | 77.5 |
| L12 | 14.1 | ND | ND | 84.4 |
| L19 | 11.9 | ND | ND | 86.3 |
| L20 | 19.5 | ND | ND | 79.2 |

ND indicates not detected.

TABLE 5a
Weight percentages of particular TAG species.

| Sample | LLLn | LnLnO | PLLn | LLL | OLnL | PLL | POLn | OLL | OLnO |
|---|---|---|---|---|---|---|---|---|---|
| H01 | 1.1 | 0.6 | 0.6 | 2.4 | 3.6 | 1.2 | 0.8 | 10.7 | 4.3 |
| H05 | 1.3 | 0.5 | 0.7 | 3.1 | 4.5 | 1.3 | 0.8 | 12.5 | 4.8 |
| H07 | 1.3 | 0.6 | 0.4 | 2.5 | 3.9 | 1.1 | 0.8 | 11.5 | 4.8 |
| H10 | 1.3 | 0.9 | 0.7 | 2.5 | 4.0 | 1.1 | 0.6 | 11.0 | 4.7 |
| H11 | 0.6 | ND | 0.7 | 1.2 | 2.9 | 1.3 | 0.9 | 8.0 | 4.0 |
| H12 | 0.8 | 0.4 | 0.8 | 2.0 | 2.6 | 1.1 | 0.5 | 9.5 | 3.3 |
| H17 | 0.4 | ND | ND | 1.1 | 2.1 | 0.7 | 0.6 | 6.0 | 4.2 |
| H18 | 0.5 | ND | 0.5 | 1.0 | 2.4 | 0.5 | 0.3 | 6.1 | 4.3 |
| H19 | 1.0 | ND | ND | 2.3 | 2.8 | 1.1 | 0.5 | 9.7 | 3.6 |
| H20 | ND | ND | 0.6 | 0.9 | 1.9 | 0.9 | 0.9 | 5.1 | 3.8 |
| L05 | 1.2 | 0.7 | 0.7 | 2.7 | 4.4 | 1.0 | 0.5 | 12.6 | 4.5 |
| L10 | 1.3 | 0.9 | 0.7 | 3.0 | 4.7 | ND | 0.8 | 13.6 | 4.6 |
| L07 | 1.4 | 0.6 | 0.6 | 3.3 | 4.9 | 1.3 | 0.7 | 14.1 | 4.4 |
| L17 | 0.9 | 0.6 | 0.8 | 1.6 | 3.4 | 0.9 | 0.7 | 9.2 | 5.1 |
| L18 | 0.6 | 0.5 | 0.8 | 1.5 | 3.5 | 0.9 | 0.7 | 8.9 | 4.8 |
| L01 | 1.1 | 0.6 | 0.8 | 3.0 | 4.1 | 1.6 | 0.7 | 13.8 | 3.9 |
| L11 | 0.9 | 0.8 | 0.8 | 1.2 | 3.5 | 1.6 | 1.1 | 8.5 | 4.8 |
| L12 | 1.0 | 0.5 | 0.7 | 2.5 | 3.7 | 1.2 | 0.5 | 12.5 | 4.0 |
| L19 | 1.3 | 0.8 | 0.8 | 3.2 | 4.2 | 1.0 | 0.5 | 13.1 | 4.1 |
| L20 | 0.5 | 0.5 | 0.4 | 0.8 | 2.4 | 1.1 | 0.8 | 6.7 | 3.8 |

ND means not detected.

TABLE 5b
Weight percentages of additional TAG species.

| Sample | OOL | POO | OOO | LOS | OGL | SOO | OOG | OLA | AOO | BOO, OON |
|---|---|---|---|---|---|---|---|---|---|---|
| H01 | 28.0 | 4.5 | 29.3 | 1.5 | 1.7 | 1.5 | 1.9 | 1.0 | 0.6 | 0.5 |
| H05 | 30.2 | 3.4 | 27.0 | 1.6 | 2.0 | 1.2 | 1.3 | ND | 0.5 | ND |
| H07 | 28.8 | 3.8 | 28.6 | 1.7 | 2.2 | 1.4 | 1.7 | 0.6 | 0.7 | 0.4 |
| H10 | 28.0 | 3.9 | 30.0 | 1.6 | 1.4 | 1.7 | 1.5 | 0.6 | 0.7 | 0.3 |
| H11 | 26.5 | 6.5 | 31.3 | 2.3 | 1.6 | 3.0 | 1.5 | 1.2 | 0.8 | 0.4 |
| H12 | 29.2 | 4.5 | 32.0 | 1.2 | 2.0 | 1.7 | 2.3 | 0.9 | 0.7 | 0.6 |
| H17 | 25.6 | 5.0 | 40.4 | 1.3 | 2.1 | 2.0 | 2.8 | 0.9 | 0.8 | 0.7 |
| H18 | 26.3 | 5.1 | 40.0 | 1.3 | 2.2 | 1.7 | 2.6 | 0.7 | 0.8 | 0.7 |
| H19 | 29.0 | 4.4 | 33.7 | 1.0 | 1.5 | 1.6 | 2.4 | 0.6 | 0.8 | 0.7 |
| H20 | 23.7 | 7.6 | 39.8 | 1.2 | 1.7 | 2.6 | 2.1 | 0.8 | 1.0 | 0.8 |
| L05 | 29.1 | 3.0 | 29.3 | 1.8 | 1.4 | 1.8 | 1.4 | ND | 0.7 | 0.5 |
| L10 | 29.8 | 3.0 | 25.7 | 1.5 | 1.6 | 1.8 | 1.5 | 0.9 | 0.7 | 0.4 |
| L07 | 29.3 | 3.2 | 24.8 | 1.2 | 1.8 | 1.2 | 1.6 | 0.9 | 0.6 | 0.6 |
| L17 | 29.6 | 3.7 | 32.6 | 0.9 | 2.1 | 1.3 | 2.0 | 0.6 | 0.6 | 0.6 |
| L18 | 29.1 | 3.8 | 31.6 | 1.2 | 2.3 | 1.5 | 2.5 | 1.1 | 0.7 | 0.8 |
| L01 | 28.7 | 3.7 | 24.9 | 1.4 | 2.1 | 1.4 | 1.8 | 0.9 | 0.7 | 0.5 |
| L11 | 26.8 | 5.7 | 29.5 | 2.2 | 1.2 | 2.7 | 1.2 | 1.0 | 0.8 | 0.3 |
| L12 | 30.0 | 3.8 | 27.0 | 1.5 | 2.5 | 1.0 | 1.7 | 0.8 | 0.6 | 0.6 |
| L19 | 30.5 | 3.0 | 25.6 | 1.3 | 2.6 | 1.3 | 2.0 | ND | 0.6 | 0.5 |
| L20 | 26.2 | 6.5 | 34.9 | 1.3 | 1.8 | 2.3 | 2.0 | 1.2 | 0.9 | 0.5 |

ND means not detected.

### Example 4

### Overall Lipid Content of Oils

The total lipid classes of the oil samples were also analysed by gas chromatography with flame ionization detection (GC/FID) and results are shown in Table 6 below as un-normalized w/w percentages based on the total oils sample lipid weight.

Quantitation of each class of compounds (e.g., tocopherols, sterols, etc.) used multi-level calibration curves with an appropriate standard (e.g., a-tocopherol, cholesterol, etc.). The quantitation of the TAG and DAG may be underestimated in this analysis due to the thermal decomposition of highly unsaturated compounds at the temperatures required to elute from the GC column. The sample size was approximately 10 mg. An internal standard (IS) of heptadecanyl stearate (HDS) was used at 1 mg. Samples were silylated with N,O,-bis-(trimethylsilyl) trifluoroacetoamide (BSTFA) with 1% trimethylchlorosilane and pyridine. The trimethylsilane (TMS) ethers were analyzed by cool on-column (COC) gas chromatography (GC) with a non-polar column stationary phase (15 m×0.25 mm×0.10 mm df, DBTM-5HT) coupled to a flame ionization detector (FID). The temperature program was 110° C. (0.2 min) to 140° C. at 30° C/min to 340° C. at 10° C/min (13.8 min). Hydrogen was the carrier gas, and inlet pressure was 6.7 psi at 110° C. in the constant flow mode. The detector temperature was 370° C. The FID air flow rate was 450 mL/min, the FID hydrogen flow rate was 40 mL/min and the makeup gas flow was 40 mL/min.

In Table 6 below, FFA stands for free fatty acids, MAG denotes monoglycerides, DAG/PL denotes diglycerides and phospholipids, TAG denotes triglycerides, and Toco denotes tocopherols. ND indicates not detected. Control samples with wild-type genotype are shaded.

TABLE 6
Lipid composition of oils (in un-normalized percentages)

| | | | | | | | Steryl |
|---|---|---|---|---|---|---|---|
| Sample | FFA | MAG | DAG/PL | TAG | Toco | Sterols | Esters |
| H01 | ND | ND | 0.87 | 101.81 | 0.11 | 0.34 | 0.77 |
| H05 | ND | ND | 0.70 | 101.64 | 0.12 | 0.34 | 0.75 |
| H07 | ND | ND | 0.60 | 98.99 | 0.10 | 0.39 | 0.99 |
| H10 | ND | ND | 0.76 | 100.72 | 0.08 | 0.34 | 0.84 |
| H11 | ND | ND | 0.68 | 103.04 | 0.11 | 0.34 | 0.68 |
| H12 | ND | ND | 0.65 | 101.71 | 0.10 | 0.38 | 0.58 |
| H17 | 0.24 | ND | 0.70 | 99.52 | 0.10 | 0.39 | 0.66 |
| H18 | ND | ND | 0.59 | 100.31 | 0.09 | 0.40 | 0.58 |
| H19 | 0.13 | ND | 0.63 | 99.43 | 0.10 | 0.42 | 0.66 |
| H20 | ND | ND | 0.68 | 101.23 | 0.14 | 0.37 | 0.62 |
| L05 | 0.28 | ND | 0.81 | 98.49 | 0.08 | 0.40 | 0.87 |
| L10 | ND | ND | 0.73 | 100.09 | 0.06 | 0.33 | 0.94 |
| L07 | 0.25 | ND | 0.78 | 98.69 | 0.07 | 0.38 | 0.99 |
| L17 | ND | ND | 0.61 | 100.77 | 0.08 | 0.36 | 0.74 |
| L18 | ND | ND | 0.63 | 100.90 | 0.09 | 0.42 | 0.75 |
| L01 | ND | ND | 0.79 | 100.23 | 0.10 | 0.32 | 0.96 |
| L11 | ND | ND | 0.77 | 101.97 | 0.08 | 0.34 | 0.86 |
| L12 | 0.24 | ND | 0.78 | 98.30 | 0.05 | 0.35 | 0.83 |
| L19 | ND | ND | 0.68 | 99.65 | 0.09 | 0.39 | 0.88 |
| L20 | ND | ND | 0.81 | 100.08 | 0.10 | 0.36 | 0.64 |

### Example 5

### Phospholipid Profile Analysis

Phospholipid Enrichment: Oil samples were dissolved in 3.0 mL of hexane and applied to a pre-conditioned zirconium-based solid phase extraction (SPE) cartridge. The sample was aspirated through the sorbent with the application of a vacuum at 12 in Hg. SPE cartridges were then washed with 4 mL each of hexane, isopropanol, and methanol. Phospholipids were eluted with two 1.5 mL aliquots of methanol with 5% ammonium hydroxide. Eluate was evaporated to dryness and reconstituted in 3 mL of 3:1 (v/v) hexane:isopropanol. 800 uL of reconstituted eluate was retained for HPLC analysis to allow for quantitation of phospholipid classes.

Separation of Phospholipid Species: The remaining about 2.2 mL of reconstituted eluate was aspirated through a pre-conditioned aminopropyl solid phase extraction cartridge. Phospholipid classes were eluted sequentially with the following solvents: 2:1 Acetonitrile:2-propanol (PC); Methanol (PE); 4:1 Isopropanol:3M HCl in methanol (PS); 2:1 chloroform:methanol with 0.3% fuming HCl (PI). Eluent was evaporated to dryness and stored until further analysis.

Quantitation of Phospholipid Species: Quantitation was performed using an Agilent Technologies HPLC column with refractive index detection. The eluate was injected onto a 150 cm long aminopropyl column. Separation was performed using 52.5:47.5 (v/v) acetonitrile:methanol as the mobile phase at a flow rate of 1.0 mL/min. Phospholipid standards (PC, PE, PI, PS, PA) dissolved in acetonitrile were used to create a calibration curve for each phospholipid class. In each case, correlation coefficients were >0.975 over 2.5 orders of magnitude. Calibration curves were run daily when analyzing samples.

FAME Analysis Sample Preparation: Samples were reconstituted in 0.75 mL 3:1 hexane:methanol. 1.0 mL of IN methanolic KOH was added to each sample. The samples were mixed thoroughly and placed in a 60 degree Celsius water bath for 90 seconds. After 90 seconds, the samples were removed from the water bath and 4.0 mL of saturated sodium chloride was added. 0.75 mL of isooctane was added to each sample. The samples were mixed thoroughly and then briefly centrifuged. The top layer was transferred to a gc vial and stored at 2 degrees Celsius pending analysis.

Instrument conditions for FAME analysis:

Column: Agilent DB-23, 5.0m×180 um×0.20 um

Oven profile: 200 degrees C. to 260 degrees C. at 2.5 degrees/min

Carrier gas: Hydrogen

Injection: 1 uL injection volume, Split, 40:1

Detection: FID, detector temp 250 degrees C.

Table 7 shows results of the phospholipid analysis. In the table below, PC stands for phosphatidyl choline, PE for phosphatidyl ethanolamine, and PI for phosphatidyl inositol. The “% SAT PL” in each of the PC, PE, and PI fractions is the percentage of saturated fatty acids out of the total fatty acids found in each of the PC, PE, and PI fractions. Also provided for comparison is the weight percentage of saturated fatty acids in the oil sample. Samples with a wild-type genotype are shaded in grey.

Table 7 shows that the percentage of saturated fatty acids found in each PL fraction in the control and experimental samples is roughly the same even though the experimental samples have a lower overall saturated fatty acid content than the controls. As phospholipids are critical components of cellular membranes, these data indicate that the saturated fatty acid content may be significantly reduced without compromising the structure of cell membranes in the plant seeds.

TABLE 7
Phospholipid analysis results.

| | % SAT PL | % SAT PL | % SAT PL | SAT |
|---|---|---|---|---|
| | PC | PE | PI | OIL |
| H1 | 11.9 | 26.1 | 25.7 | 5.4 |
| H5 | 12.1 | 27.1 | 21.5 | 4.1 |
| H7 | 12.1 | 28.3 | 20.5 | 4.3 |
| H10 | 10.7 | 28.8 | 20.4 | 4.8 |
| H11 | 11.5 | 27.1 | 22.7 | 7.7 |
| H12 | 12.0 | 24.6 | 22.8 | 4.9 |
| H17 | 11.5 | 30.1 | 22.5 | 4.7 |
| H18 | 12.0 | 27.1 | 19.8 | 4.6 |
| H19 | | | | 4.6 |
| H20 | 11.4 | 27.3 | 23.9 | 7.3 |
| L1 | 12.4 | 31.6 | 20.7 | 5.5 |
| L5 | 11.3 | 28.1 | 29.2 | 4.3 |
| L7 | 11.8 | 30.2 | 17.3 | 4.2 |
| L10 | | | | 4.6 |
| L11 | 12.2 | 28.0 | 20.4 | 7.8 |
| L12 | 11.4 | 27.1 | 18.7 | 4.5 |
| L17 | 11.3 | 30.7 | 24.2 | 3.9 |
| L18 | 12.2 | 28.6 | 21.4 | 4.0 |
| L19 | 12.7 | 24.4 | 20.4 | 3.8 |
| L20 | 11.4 | 26.5 | 19.8 | 7.1 |

What is claimed is:

1-30 (canceled)

31. A canola oil comprising triacylglycerols (TAGs), wherein 11-16% of the TAGs comprise one saturated fatty acid and two unsaturated fatty acids and wherein 82-88% of the TAGs comprise three unsaturated fatty acids, and wherein 81-91% of the TAGs comprise at least one oleic acid and wherein 7-12% of the TAGs comprise at least one linolenic acid.

32. The canola oil of claim 31, wherein the oil comprises 3.5% to 5.5% saturated fatty acids.

33. The canola oil of claim 32, wherein the oil comprises 3.5% to 4.5% saturated fatty acids.

34. The canola oil of claim 32, wherein the oil comprises 62-74% oleic acid and 2.5-5.0% linolenic acid.

35. The canola oil of claim 32, wherein 11-13% of TAGs in the oil comprise one saturated fatty acid and two unsaturated fatty acids.

36. The canola oil of claim 32, wherein the oil does not contain detectable levels of TAGs comprising two or three saturated fatty acids.

37. The canola oil of claim 32, wherein 3-5% of TAGs comprise two oleic acids and one palmitic acid.

38. The canola oil of claim 32 wherein 1-2% of TAGs comprise two oleic acids and one stearic acid.

39. The canola oil of claim 32, wherein 1-3% of TAGs comprise three linoleic acids.

40. The canola oil of claim 32, wherein 10-14% of TAGs comprise one oleic acid and two linoleic acids.

41. The canola oil of claim 31, wherein the oil comprises phospholipids including phosphatidyl choline, phosphatidyl ethanolamine, and phosphatidyl inositol, and wherein 10-13% of fatty acids found in the phosphatidyl choline are saturated, 24-31% of fatty acids found in the phosphatidyl ethanolamine are saturated, and/or 17-30% of the phospholipids found in the phostphatidyl inositol are saturated.

42. The canola oil of claim 32, wherein the oil has been degummed, subjected to alkali or physical refinement, bleached, deodorized, and/or dewaxed.

43. The canola oil of claim 31, wherein the oil is prepared from seeds of a Brassica plant comprising a mutant allele at a fatty acyl-acyl carrier protein thioesterase A2 (FATA2) locus, wherein the mutant allele results in production of a FATA2 polypeptide having reduced thioesterase activity relative to a corresponding wild-type FATA2 polypeptide.

44. The canola oil of claim 43, wherein the Brassica plant further comprises a mutation at the chromosome N01 quantitative trait locus 1 (QTL 1) allele and a mutation at the chromosome N19 QTL2 allele.

45. The canola oil of claim 43, wherein the Brassica plant further comprises a mutant allele at a fatty acyl-acyl carrier protein thioesterase l (FATB) locus, such as at any combination of the FATB1, FATB2, FATB3, and FATB4 loci, wherein the mutant allele(s) results in the production of a FATB polypeptide having reduced thioeterase activity relative to a corresponding wild-type FATB polypeptide.

46. The canola oil of claim 43, wherein the Brassica plant further comprises a mutant allele at a FAD2 locus.

47. The canola oil of claim 43, wherein the Brassica plant further comprises a mutant allele at a FADS locus, such as at a FAD3A, FAD3B, FAD3D, or FAD3F locus.

48. A seed of a Brassica plant of claim 43.

49. A Brassica plant producing the oil of claim 31

50. A food composition prepared from the oil of claim 31.


SEQUENCE LISTING (17 sequences)

SEQ ID NO: 1 - DNA, 1557 bp, Brassica napus
atggtggctacttgcgctacgtcgtcgttttttcatgttccatcttcttcctcgcttgatactaatgggaaggggaacagagttgggtctactaattttgctggacttaactcaacgccaagctctgggaggatgaaggttaagccaaacgcttaggctccacccaagatcaacgggaagaaagctaacttgcctggctctgtagagatatcaaagtctgacaacgagacttcgcaacccgcacacgcaccgaggacgtttatcaaccagctacctgactggagcatgcttcttgctgccataacaactattttcttagcggcggagaaacagtggatgatgcttgactggaaacctaggcgttctgatatgattatggatcctttcggtttagggagaatcgttcaggatggtcttgtgttccgtcagaatttttccattaggtcttatgagataggtgctgatcgctctgcgtctatagaaactgtcatgaatcatttacaggtactgctttgattgtggttacactcacatgttgtcccaatagatatatgctcatgacaagctcttatgctaatgacaggaaacggcgcttaatcatgtgaagtctgccggactgctggaaaatgggtttgggtccactcctgagatgtttaagaagaatttgatatgggtcgttgctcgtatgcaggttgtcgttgataaatatcctacttggtaagccattgttagtcttagcacttgacttaaaatcattttgcatattacagtgtgcgtagatcatttgcttattcaaatatctgactcacaggggagatgttgtggaagtggatacttgggttagtcagtctggaaagaatggtatgcgtcgtgattggctagttcgggattgcaatactggagaaattgtaacgcgagcatcaaggtcagagttcttatattttggtttactccagctattatcgttttgctctctgtttgtattgtttcctctgccattagtttgataattgagtctttatagttgtatatgtatggcaattttcttctttttgcagtttgtgggtgatgatgaataaactcacaaggagattgtcaaagattcctgaagaggttcgaggggaaatagagccttattttgtgaactctgatcctgtcattgccgaagacagcagaaagttaacaaaacttgatgacaagactgctgactatgttcgttctggtctcactgtaagtaccttacctttcgacaagcctgtcaaaactcttgaggttctaatggtttggtaatgaacttttttttggcagccgaggtggagtgacttggatgttaaccagcatgttaacaatgtaaagtacattgggtggatactggagagtgctccagcagggatgctggagagtcagaagctgaaaagcatgactctggagtatcgcagggagtgcgggagagacagtgtgcttcagtctctcaccgcagtctctggatgtgatgtcggtaacctcgggacagccggggaagtggagtgtcagcatttgcttcgactccaggatgga

SEQ ID NO: 2 - DNA, 1572 bp, Brassica napus
atggtggccacctcagctacatcctcattcttccctctcccatcttcccccctcgaccccaccgcaaaaaccaacaaagtcaccacctccaccaacttctccggcctcacacccacgccgaactccgccaggatgaaggttaaaccaaacgctcaggccccacccaagatcaacggcaagagagtcggcctccctggctcggtggagatcttgaagcctgatagcgagacttcgcaaccagcaccgaggacgttcatcaaccagctgcctgactggagcatgctcctcgccgccatcacgaccgtcttcttggcggctgagaagcagtggatgatgctcgactggaaaccgaggcgttctgacgtgattatggatccgtttgggttagggaggatcgttcaggatgggcttgtgttccgtcagaatttttctattcggtcttatgagataggtgctgatcgctctgcgtctatagaaacggttatgaatcatttacaggtactgattatgattatgattgtagtcgcttgttgttactggacaaacttaaatatgtattgctcttatggttgtgataggaaacggcactcaaccatgttaagactgctgggctgcttggagatgggtttggttctactcctgagatggttaagaagaacttgatatgggttgttactcgtatgtaggttgtcgttgataaatatcctacttggtaagctattctcaaacaactctgagaatcactgcttcctttgtgagtcatttgcttattcaaatatctgcctcataggggagatgttgtggaagtagatacatgggtgagccagtctggaaagaacggtatgcgtcgtgattggcttgttcgggatggcaatactggagagattttaacaagagcatcaaggttagattttattttttggtttacttgggttagatatctgataattgagttataatcatctccgtgttgtgtaaactattctttttgcagtgtgtgggtgatgatgaataaactgacaagaagattatcaaagattcctgaagaggttcgaggggagatagagccttactttgttaactcagacccagtccttgccgaggacagcagaaagttaacaaaacttgatgacaaaactgctgtctatgttcgttctggtctcactgtaagtacaaatacttcactctatgtttcaacaaagcctgtaaatttttgagtctcttacaggtttggtaatgaactttttgcagccgcgttggagtgacttggatgttaaccagcacgttaacaatgtgaagtacatcgggtggatactggagagtgctccagtggggatgatggagagtcagaagctgaaaagcatgactctggagtatcgcagggagtgtgggagagacagtgtgctccagtccctcaccgcggtttcgggctgcgatatcggtagcctcgggacagccggtgaagtggaatgtcagcatctgctcagactccaggatggagccgaagtggtgagaggaagaacagagtggagttccaaaacatcaacaacaacttgggacatcacaccgtga

SEQ ID NO: 3 - DNA, 1500 bp, Brassica napus
atggtggccacctcagctacatcctcattcttccctctcccatcttcccccctcgaccccaccgcaaaaaccaacaaagtcaccacctccaccaacttctccggcctcacacccacgccgaactccgccaggatgaaggttaaaccaaacgctcaggccccacccaagatcaacggcaagagagtcggcctccctggctcggtggagatcttgaagcctgatagcgagacttcgcaaccagcaccgaggacgttcatcaaccagctgcctgactgaagcatgctcctcgccgccatcacgaccgtcttcttggcggctgagaagcagtggatgatgctcgactggaaaccgaggcgttctgacgtgattatggatccgtttgggttagggaggatcgttcaggatgggcttgtgttccgtcagaatttttctattcggtcttatgagataggtgctgatcgctctgcgtctatagaaacggttatgaatcatttacaggtactgattatgattatgattgtagtcgcttgttgttactggacaaacttaaatatgtattgctcttatggttgtgataggaaacggcactcaaccatgttaagactgctgggctgcttggagatgggtttggttctactcctgagatggttaagaagaacttgatatgggttgttactcgtatgcaggttgtcgttgataaatatcctacttggtaagctattctcaaacaactctgagaatcactgcttcctttgtgagtcatttgcttattcaaatatctgcctcataggggagatgttgtggaagtagatacatgggtgagccagtctggaaagaacggtatgcgtcgtgattggcttgttcgggatggcaatactggagagattttaacaagagcatcaaggttagattttattttttggtttacttgggttagatatctgataattgagttataatcatctccgtgttgtgtaaactattctttttgcagtgtgtgggtgatgatgaataaactgacaagaagattatcaaagattcctgaagaggttcgaggggagatagagccttactttgttaactcagacccagtccttgccgaggacagcagaaagttaacaaaacttgatgacaaaactgctgtctatgttcgttctggtctcactgtaagtacaaatacttcactctatgtttcaacaaagcctgtaaatttttgagtctcttacaggtttggtaatgaactttttgcagccgcgttggagtgacttggatgttaaccagcacgttaacaatgtgaagtacatcgggtggatactggagagtgctccagtggggatgatggagagtcagaagctgaaaagcatgactctggagtatcgcagggagtgtgggagagacagtgtgctccagtccctcaccgcggtttcgggctgcgatatcggtagcctcgggacagccggtgaagtggaatgtcagcatctgctcagactccaggatgga

SEQ ID NO: 4 - DNA, 1664 bp, Brassica napus
atggtggctacttccgctacgtcgtcgttttttcatgttccatcttcctcctctcttgatactaatgggaaggggaacagagttgcgtccacgaacttcgctggacttaactcaacgccaagctctgggaggatgaaggttaaaccaaacgctcaggctccacccaagatcaacgggaagaaagctaacttgcctggttctgcagagatatcaaagtctgacaacgagacttcgcaacccgcacccgcaccgaggacgtttatcaaccagctgcctgactggagcatgcttctcgctgccataacaactattttcttagcggctgagaaacagtgaatgatgcttgactggaaacccaggcgttctgatatgataatggatcctttcggtttagggagaatcgttcaggatggtcttgtgtttcgtcagaatttctccattaggtcttatgagataggtgctgatcgctctgcgtctatagaaactgttatgaatcatttacaggtaggtactactttgattgttatcacacttgtcactggacacccaatagatatatatgctcatgacaagctcttatgctaatgacaggaaacggccctaaaccatgtgaagtctgccggactgctggaaaatgggtttggttctactcccgagatgtttaagaagaacttgatatgggtcgttgctcgtatgcaggttgtcgttgataaatatcctacttggtaagccattgtcagtcttaccacttaacttaaaatcattatgcatattacagtttgcatagatcattacttattcaaatatctgactaacaggggagatgttgtggaagtggatacatgggttagtcagtccggaaagaatggtatgcgtcgtgattggctggttcgggattgcaatactggagaaattgtaacgcgagcatcaaggtcagagttcttatgttttggtttactgactccagctattatcattttgctctctgtttgtattgtttgctctgccattaatatgataatagagactttatagttgtatatgtatggcaattttcttctttttgcagtttgtgggtgatgatgaataaactgacaaggagattgtcaaagattcctgaagaggttcgtggggaaatagagccttattttgtgaactctgatcctgtcattgccgaagacagcagaaagttaacaaaactggatgacaagactgctgactatgttcgttcgggtctcactgtaagtaccctacctttcaacaagcctttaaaactcttgaggttctaatggtttggtaataaacttttttttcagccgagttggagtgacttagatgttaaccagcatgttaacaatgtaaagtacattgggtggatactggagagtgctccagcagggatgctggagagtcagaagctgaaaagcatgactctggagtatcgcagggagtgcgggagagacagtgtgcttcagtctctcaccgcggtctctggatgtgatgtcggtaacctcgggacagccggggaagtggagtgtcagcatttgcttcgtctccaggatggagctgaagtggtgagaggaagaacagctgaagtggtgagaggaagaacagagtggagttccaagatagaagcaacaacttgggacactgctacatcgtaa

SEQ ID NO: 5 - DNA, 1557 bp, Brassica napus
atggtggctacttgcgctacgtcgtcgttttttcatgttccatcttcttcctcgcttgatactaatgggaaggggaacagagttgggtctactaattttgctggacttaactcaacgccaagctctgggaggatgaaggttaagccaaacgctcaggctccacccaagatcaacgggaagaaagctaacttgcctggctctgtagagatatcaaagtctgacaacgagacttcgcaacccgcacacgcaccgaggacgtttatcaaccagctacctgactggagcatgcttcttgctgccataacaactattttcttagcggcggagaaacagtggatgatgcttgactggaaacctaggcgttctgatatgattatggatcctttcggtttagggagaatcgttcaggatggtcttgtgttccgtcagaatttttccattaggtcttatgagataggtgctgatcgctctgcgtctatagaaactgtcatgaatcatttacaggtactgctttgattgtggttacactcacatgttgtcccaatagatatatgctcatgacaagctcttatgctaatgacaggaaacggcgcttaatcatgtgaagtctgccggactgctggaaaatgggtttgggtccactcctgagatgtttaagaagaatttgatatgggtcgttgctcgtatgcaggttgtcgttgataaatatcctacttggtaagccattgttagtcttagcacttgacttaaaatcattttgcatattacagtgtgcgtagatcatttgcttattcaaatatctgactcacaggggagatgttgtggaagtggatacttgggttagtcagtctggaaagaatggtatgcgtcgtgattggctagttcgggattgcaatactggagaaattgtaacgcgagcatcaaggtcagagttcttatattttggtttactccagctattatcgttttgctctctgtttgtattgtttcctctgccattagtttgataattgagtctttatagttgtatatgtatggcaattttcttctttttgcagtttgtgggtgatgatgaataaactcacaaggagattgtcaaagattcctgaagaggttcgaggggaaatagagccttattttgtgaactctgatcctgtcattgccgaagacagcagaaagttaacaaaacttgatgacaagactgctgactatgttcgttctggtctcactgtaagtaccttacctttcgacaagcctgtcaaaactcttgaggttctaatggtttggtaatgaacttttttttggcagccgaggtggagtgacttggatgttaaccagcatgttaacaatgtaaagtacattgggtggatactggagagtgctccagcagggatgctggagagtcagaagctgaaaagcatgactctggagtatcgcagggagtgcgggagagacagtgtgcttcagtctctcaccgcagtctctggatgtgatgtcggtaacctcgggacagccggggaagtggagtgtcagcatttgcttcgactccaggatgga

SEQ ID NO: 6 - DNA, 1563 bp, Brassica napus
atggtggccacctcagctacatcctcattcttccctctcccatctttccccctcgaccccaccgcaaaaaccaacaaagtcaccacctccaccaacttctccggcctctcccccactccaaactcctccggcaggatgaaggttaaaccaaacgctcaggccccacccaagatcaacggcaagagagtcggtctcccttctggctcggtgaagcctgataacgagacgtcctcacagcatcccgcagcaccgaggacgttcatcaaccagctgcctgactggagcatgcttcttgctgcaataacaaccgtcttcttggcggctgagaagcagtggatgatgcttgactggaaaccgaggcgctctgacgtgattatggatccgtttgggttagggaggatcgttcaggatgggcttgtgttccgtcagaatttctctattcggtcttatgagataggtgctgatcgctctgcgtctatagaaacggttatgaatcatttacaggtactgattatgattatgattatgattgtagttgcttgttgttactggacaaagttaatatgtattgctgttatggttatgataggaaacggcactcaaccatgttaagactgctggactgcttggagatgggtttggttctactcctgagatggttaagaagaacttgatttgggttgttactcgtatgcaggttgtcgttgataaatatcctacttggtaagctattctcaagcaaccctgagaatcactgcttcctttgtcatttgcttattcaaatatctgtctcacaggggagatgttgtggaagtagatacatgggtgagccagtctggaaagaacggtatgcgtcgtgattggctagttcgagatggcaatactggagaaattttaacaagagcatcaaggttagatttttatttatcggttaggtatctgaaaatttgagttactaatgcaaaatattatttttgcagtgtgtgggtgatgatgaataaactgacaagaagattatcaaagattcctgaagaggttcgaggggagatagagccttactttgttaattcagacccagtccttgctgaggacagcagaaagttaactaaacttgatgacaagactgctgactatgttcgttctggtctcactgtaagtatgcatactttctctatgtttcatcaaagcctgtaaacttctgagattcttacagtttttatttggtaatttaaacttttgcagccgcgttggagtgacttggatgttaaccagcacgttaacaatgtgaagtacatcgggtggatactggagagtgcacctgtggggatgatggagagtcagaagctgaaaagcatgactctggagtatcgcagggagtgcgggagggacagtgtgcttcagtccctcaccgcggtttcgggctgcgatgttggtagtcttgggacagctggtgaagtggaatgtcagcacctgctccgtctccaggatggagctgaagtggtgagaggaagaacagagtggagttccaaaacatcaacaacaacttgggacattacaccgtga

SEQ ID NO: 7 - DNA, 1572 bp, Brassica napus
atggtggccacctcagctacatcctcattcttccctctcccatcttcccccctcgaccccaccgcaaaaaccaacaaagtcaccacctccaccaacttctccggcctcacacccacgccgaactccgccaggatgaaggttaaaccaaacgctcaggccccacccaagatcaacggcaagagagtcggcctccctggctcggtggagatcttgaagcctgatagcgagacttcgcaaccagcaccgaggacgttcatcaaccagctgcctgactggagcatgctcctcgccgccatcacgaccgtcttcttggcggctgagaagcagtggatgatgctcgactggaaaccgaggcgttctgacgtgattatggatccgtttgggttagggaggatcgttcaggatgggcttgtgttccgtcagaatttttctattcggtcttatgagataggtgctgatcgctctgcgtctatagaaacggttatgaatcatttacaggtactgattatgattatgattgtagtcgcttgttgttactggacaaacttaaatatgtattgctcttatggttgtgataggaaacggcactcaaccatgttaagactgctgggctgcttggagatgggtttggttctactcctgagatggttaagaagaacttgatatgggttgttactcgtatgcaggttgtcgttgataaatatcctacttggtaagctattctcaaacaactctgagaatcactgcttcctttgtgagtcatttgcttattcaaatatctgcctcataggggagatgttgtggaagtagatacatgggtgagccagtctggaaagaacggtatgcgtcgtgattggcttgttcgggatggcaatactggagagattttaacaagagcatcaaggttagattttattttttggtttacttgggttagatatctgataattgagttataatcatctccgtgttgtgtaaactattctttttgcagtgtgtgggtgatgatgaataaactgacaagaagattatcaaagattcctgaagaggttcgaggggagatagagccttactttgttaactcagacccagtccttgccgaggacagcagaaagttaacaaaacttgatgacaaaactgctgtctatgttcgttctggtctcactgtaagtacaaatacttcactctatgtttcaacaaagcctgtaaatttttgagtctcttacaggtttggtaatgaactttttgcagccgcgttggagtgacttggatgttaaccagcacgttaacaatgtgaagtacatcgggtggatactggagagtgctccagtggggatgatggagagtcagaagctgaaaagcatgactctggagtatcgcagggagtgtgggagagacagtgtgctccagtccctcaccgcggtttcgggctgcgatatcggtagcctcgggacagccggtgaagtggaatgtcagcatctgctcagactccaggatggagccgaagtggtgagaggaagaacagagtggagttccaaaacatcaacaacaacttgggacatcacaccgtga

SEQ ID NO: 8 - DNA, 1664 bp, Brassica napus
atggtggctacttccgctacgtcgtcgttttttcatgttccatcttcctcctctcttgatactaatgggaaggggaacagagttgcgtccacgaacttcgctggacttaactcaacgccaagctctgggaggatgaaggttaaaccaaacgctcaggctccacccaagatcaacgggaagaaagctaacttgcctggttctgcagagatatcaaagtctgacaacgagacttcgcaacccgcacccgcaccgaggacgtttatcaaccagctgcctgactggagcatgcttctcgctgccataacaactattttcttagcggctgagaaacagtggatgatgcttgactggaaacccaggcgttctgatatgataatggatcctttcggtttagggagaatcgttcaggatggtcttgtgtttcgtcagaatttctccattaggtcttatgagataggtgctgatcgctctgcgtctatagaaactgttatgaatcatttacaggtaggtactactttgattgttatcacacttgtcactggacacccaatagatatatatgctcatgacaagctcttatgctaatgacaggaaacggccctaaaccatgtgaagtctgccggactgctggaaaatgggtttggttctactcccgagatgtttaagaagaacttgatatgggtcgttgctcgtatgcaggttgtcgttgataaatatcctacttggtaagccattgtcagtcttaccacttaacttaaaatcattatgcatattacagtttgcatagatcattacttattcaaatatctgactaacaggggagatgttgtggaagtggatacatgggttagtcagtccggaaagaatggtatgcgtcgtgattggctggttcgggattgcaatactggagaaattgtaacgcgagcatcaaggtcagagttcttatgttttggtttactgactccagctattatcattttgctctctgtttgtattgtttgctctgccattaatatgataatagagactttatagttgtatatgtatggcaattttcttctttttgcagtttgtgggtgatgatgaataaactgacaaggagattgtcaaagattcctgaagaggttcgtggggaaatagagccttattttgtgaactctgatcctgtcattgccgaagacagcagaaagttaacaaaactggatgacaagactgctgactatgttcgttcgggtctcactgtaagtaccctacctttcaacaagcctttaaaactcttgaggttctaatggtttggtaataaacttttttttcagccgagttggagtgacttagatgttaaccagcatgttaacaatgtaaagtacattgggtggatactggagagtgctccagcagggatgctggagagtcagaagctgaaaagcatgactctggagtatcgcagggagtgcgggagagacagtgtgcttcagtctctcaccgcggtctctggatgtgatgtcggtaacctcgggacagccggggaagtggagtgtcagcatttgcttcgtctccaggatggagctgaagtggtgagaggaagaacagctgaagtggtgagaggaagaacagagtggagttccaagatagaagcaacaacttgggacactgctacatcgtaa

SEQ ID NO: 9 - DNA, 1714 bp, Brassica napus
atggtggccacctctgctacatcctcattcttccctctcccatcttcctctctcgaccccaatggcaaaaccaacaaagccacctccaccaacttctccggactcaaccccacaccaaactcttccggcaggttaaaggtcaaaccaaacgctcaggctccatccaagatcaacggcaagaaagtctccttgccaggctcagtacacatcgtaaagactgataataaccacgatctctcgcaacaaaacgcacccagaacgttcatcaaccagctacctgactggagcatgcttctcgccgccatcacaacggtcttcttagcagctgagaagcagtggatgatgcttgatactaaaccgagacgctccgacatgattatggatccgtttgggttagggagaatcgttcaggatgggcttgtgtaccgtcagaatttcgatatcaggtcttatgaaataggtgctgatcgctctgcatctatagaaactgtcatgaatcacttacaggtatattacaatcacactcgtttgatactatagcttgacccgcactgatgttggtttttatatttttataaattgtttagtgacatatagatataggttatttagatatttctaggttcctacgaacctacccggactcaaaccctgtccgtaaaattgagtttaattttaaaccaaaaaaatccgatacccgaaaaaaccgatctgtatctaactcttgtcctcatgacaggaaacggctctcaaccatgtgaagtctgcaggactgctgggagatgggtttggttctacacctgagatggttaagaagaacttgatatgggttgttactcgtatgcaggttgtagttgataaatatcctacttggtaagctctcttgccacttaaccttaaacaatatgcatgaatcatttgcttattcaaatgtctgtttcaccaggggagatgttgttgaagtagatacatgggtcagtaagtctgggaagaatggtatgcgtcgtgattggctagttcgtgattgcaatactggagaaatcttaacacgcgcatcaaggttagctttattttgtttttgtttactccagctattatctgattattgagttataaccatctctatgttacaaaacagtgtgtgggtgatgatgaataaactgacaaggagattatcaaagcttcctgaagaggttcgaggggaaatagagccttactttgtgaactctgacccaatccttgccgaggacagcagaaagttaacaaagctagatgacaagactgctgactatgttcgctctggtctcaccgtaagtataaatattcaactctttatcttttagcgtgtaaaactcttgagagattcttatgagtttggtgatgaacttttgcagccgagatggagtgacttggatgttaaccagcatgttaacaacgtgaagtacattggttggatactcgagagtgctccagtagagatgatggagaagcataagctgaaaagcatgactctggagtataggagggaatgcgggagagacagtgtgcttcagtctctcaccgcggtttcgggatgcgatgttggtagcctcgggacagctggtgaagtggaatgtcagcatttgcttcgacaccaggatggagctgaagtggtgaagggacgaacagtgtggagttcgaaaacaccatcaacaacttgggacactacatcgta

SEQ ID NO: 10 - DNA, 1891 bp, Brassica napus
atggtggccacctctgctacatcctcattcttccctctcccatcttcctctctcgaccctaatggcaaaaccaacaaactcacctccaccaacttctctggactcaaccccataccaaactcttccggcaggttaaaggtcaaaccaaacgcccaagctccatccaagatcaacggcaataatgtctccttgccaggctcagtacacatcgtaaagactgataataaccacgatctctcgcaacaacacgcacccagaacgttcatcaaccagctacctgactggagcatgcttctcgccgccatcacaacggtcttcttagctgctgagaaacagtggatgatgcttgactcgaaaccgaggcgttctgatatgattatggatccgttcgggttagggaggatcgttcaggatgggcttgtgtaccgtcagaacttcgatatcaggtcttatgaaataggtgctgatcgctctgcgtctatagaaacagtcatgaaccacttacaggtatattacaatcacactcgattgatactagagcttgacatgttggtttttatctttttataaattgtttagtgacattttcaaacatatagatataggttatttagatatttctaggttcctacaaacctacccagactcaaaccccgtccggaaatttataatattaataccgaacagagttttattttaaaccaaaaaatcagttgacccgcacgggatgttggtttttatctattttatacattgtttaaggacatttttaaacatataaatataggttatttagatatttctaggttcctacgaacctacccggaaatttataatacccgaacatagtttaatttttaaaccaaaaaatccaatacccgaaaaaaccaatctgtgatatgcatgatctaactcttgtcctcgtgacaggaaacggctctcaaccatgtgaagtctgctggactgctgggagatgggtttggttctacccctgagatggttaagaagaacttgatatgggtcgttactcgtatgcaggttgtcgttgataaatatcctacttggtaagccctcttagcacttaaccttaaaacaatatgcatgaatcatttgcttattcaaatgtctgcttcaccaggggagatgttgttgaagtagatacatgggttagtaagtctgggaagartggtatgcgtcgtgattggcttgttcgggattgtaatactggagaaattttaacaagagcatcaaggttagcttctttttgtttactccagctattatctgattattgagttataaccatctctgtgttgcaaaacagtgtgtgggtgatgatgaataaagtgacaaggagattatcaaagcttcctgaagaggttcgaggggaaatagagccttactttgtgaactctgaccctatccttgccgaggacagcagaaagttaacaaaactagatgagaagactgctgactatgttcgctctggtctcaccgtaagtataaatatttgtttttatctttcagcaagtgagattctgatgggtttggtgattatctaacttttgcagccgagatggagtgacttggatgttaaccagcatgttaacaacgtgaagtacattggttggatactcgagagtgctccagtggagatgatggagaagcataagctgaaaagcatgactctggagtataggagggaatgcgggagagacagtgtgcttcagtctctcaccgcggtttcgggttgcgatgttggtagcctcgggacagctggtgaagtggaatgtcagcatttgcttcgactccaggatggagctgaagtggtgaagggacgaacagtgtggagttccaaaacaccatcaacaacttgggacactacatcgta

SEQ ID NO: 11 - DNA, 1163 bp, Brassica napus
aagtgtggattctcgacggatggatttgccacaacactcaccatgaggaaattgcatctatatgggtcactgcaagaatgcacattgagatctacaagtacccagcttggtattttcttttcttaggcttctttgactagttgacactttagaggtcggagtttgtaaacctcagagctttttattacttggttaacaggagtgatgttgttgagatagagacatggtgccagagtgaaggaaggattggaacgagacgtgattggattctaagggactctgctacaaatgaagttattgggcgtgctacaaggtttgccaaaaacagatttgttactactattcataaattcatttttttatctgccttcaatcaatataataatgcaaatcactgacattagtcgcacaacagtaactcccatatacgttgcttatttagttataaagacttatgcatattctggaacctgagcttgtttttgtttgacaaatgttacatgggtcttacagcaagtgggtgatgatgaaccaagacacaaggcggcttcaaagagttacagatgaagttcgggacgagtacttggttttctgtcctcgagaacccaggtgaagaagaatcatcatgcttcccttataattgctagttaaacagttaatatttaagcatgtggatctcaacctgttgtcctctgtatttctcgtagactagcgtttccagaagagaacaatagcagcttaaagaaaatcccaaaactagaagatccagctcagtattctatgctagagcttaagcttcggcgagctgatctggacatgaaccagcacgtgaataacgtcacctacattggatgggtgcttgaggtgagtaccttaataaagcctacaaaacgtctatcattttaatcatacatatgagctaactaactattaaatttgagtttggttccctggtaatggcagagcatacctcaagaaatcattgatacgcatgagcttcaagttataactctagattacagaagagaatgccagcaagatgacattgtagattcactcaccacctctgaaatccctgacgacccgatctcaaagcttaccgggaccaacggatctgccacgtcaagcatacaaggacacaatgagagccagttcttgcatat

SEQ ID NO: 12 - protein, 36 aa, Brassica napus
LEDPAQYSMLELKPRRADLDMNQHVNNVTYIGWVLE

SEQ ID NO: 13 - protein, 367 aa, Arabidopsis thaliana
MLKLSCNVTDHIHNLFSNSRRIFVPVHRQTRPISCFQLKKEPLRAILSADHGNSSVRVADTVSGTSPADRLRFGRLMEDGFSYKEKFIVRSYEVGINKTATIETIANLLQEVACNHVQNVGFSTDGFATTLTMRKLHLIWVTARMHIEIYKYPAWSDVVEIETWCQSEGRIGTRRDWILKDCATGEVIGRATSKWVMMNQDTRRLQRVTDEVRDEYLVFCPPEPRLAFPEENNSSLKKIPKLEDPAQYSMLGLKPRRADLDMNQHVNNVTYIGWVLESIPQEIIDTHELKVITLDYRRECQQDDIVDSLTTSETPNEVVSKLTGTNGSTTSSKREHNESHFLHILRLSENGQEINRGRTQWRKKSSR

SEQ ID NO: 14 - protein, 223 aa, Brassica napus
GFSTDGFATTLTMRKLHLIWVTARMHIEIYKYPAWSDVVEIETWCQSEGRIGTRRDWILRDSATNEVIGRATSKWVMMNQDTRRLQRVTDEVRDEYLVFCPREPRLAFPEENNSSLKKIPKLEDPAQYSMLELKPRRADLDMNQHVNNVTYIGWVLESIPQEIIDTHELQVITLDYRRECQQDDIVDSLTTSEIPDDPISKLTGTNGSATSSIQGHNESQFLH

SEQ ID NO: 15 - DNA, 1163 bp, Brassica napus
aagtgtggattctcgacggatggatttgccacaacactcaccatgaggaaattgcatctcatatgggtcactgcaagaatgcacattgagatctacaagtacccagcttggtattttcttttcttaggcttctttgactagttgacactttagaggtcggagtttgtaaacctcagagctttttattacttggttaacaggagtgatgttgttgagatagagacatggtgccagagtgaaggaaggattggaacgagacgtgattggattctaagggactctgctacaaatgaagttattgggcgtgctacaaggtttgccaaaaacagatttgttactactattcataaattcatttttttatctgccttcaatcaatataataatgcaaatcactgacattagtcgcacaacagtaactcccatatacgttgcttatttagttataaagacttatgcatattctggaacctgagcttgtttttgtttgacaaatgttacatgggtcttacagcaagtgggtgatgatgaaccaagacacaaggcggcttcaaagagttacagatgaagttcgggacgagtacttggttttctgtcctcgagaacccaggtgaagaagagtcatcatgcttcccttataattgctagttaaacagttaatatttaagcatgtggatctcaacctgttgttctctgtatttctcgtagactagcgtttccagaagagaacaatagcagcttaaagaaaatcccaaaactagaagatccagctcagtattctatgctagagcttaagcttcggcgagctgatctggacatgaaccagcacgtgaataacgtcacctacattggatgggtgcttgaggtgagtaccttaataaagcctacaaaacgtctatcattttaatcatacatatgagctaactaactattaaatttgagtttggttccctggtaatggcagagcatacctcaagaaatcattgatacgcatgagcttcaagttataactctagattacagaagagaatgccagcaagatgacattgtagattcactcaccacctctgaaatccctgacgacccgatctcaaagcttaccgggaccaacggatctgccacgtcaagcatacaaggacacaatgagagccagttcttgcata

SEQ ID NO: 16 - DNA, 3691 bp, Brassica napus
atggttgttgctatggaccaacgcaccaatgtgaacggagatgccggtgcccggaaggaagaagggtttgatccgagcgcacaaccgccgtttaagatcggggacataagggctgcgattcctaagcattgttgggtgaaaagtcctttgagatctatgagctacgtagccagagacatttgtgccgtcgcggctttggccattgccgccgtgtattttgatagctggttcctctgtcctctctattgggtcgcccaaggaacccttttctgggccatcttcgtcctcggccacgactggtaaagtttcttccattttgcattgcatcgatttattgaatgcacgttctacgagtattgtttgtcagttacttcgtaaaatgattcttttgatgttcattttttgaagatctaagatttttttttttagattttctttttaaatcattgttccaccaccacctttcatcggtcgtacgactcgttacaacaccacatctttattttctataattactactgcttccgcattttatggatctctcaacttataattaaagtataatatcaagaatatctattatttttcttaaacaagaaagataatattgtttctttgttattttggtgtatttccaatctatttcgagatttagaaatgtgacacgtcattaccttgttgaagtgtttaaaacaaacatggaaagtttaaataaatagtgcaataaatgatatatatgtatatgatgaataatgatgtgaaatataattgaataatggcagtggacatgggagtttctcagacattcctctgctgaatagtgtggttggccatattcttcattccttcatcctcgttccttaccatggttggtaagtcagcttatcaaccctttttactatattattaattattaaacttgcatttgtatacttggtgcaagttggtaaatgtaatctgataactgaaaatctattcattgctcgttctattttttttttggctagagacaattttataattaaataatgcatgtgagaatatgactatttatgtgaggtagcttttcttattcctgtcgaaaagcatcaaatctttagcaacgaaggaaaaaggaatcaaattttttattaaatgcaatgggtctatgtcttggtcattagttttttgcatataatttatttatatttttttcttaacagcagctaatttaattataattaaatattcattttataaataatattagaccaattattaaaggttagatattttaagaattattcatgactttgtttattggaactccttttatcttttaatcttttctatttctccatttttaataatgagaaactgacttcaaatctccaataaagatggtcttatgtagtaacagtataattttttgtttggtaaatgtaacatcatcttcaaatatctttgaaaatagacttacatgcattattttgctgcgacattattgtcacttattcctggcaataaattagtttattactgaacttttttttggtcaatttattactagtaactttaaacttaaaagagtgagattgtttgatcaaaaaaaataaaaatagagtgagatagttagaatctgccatgaaagcaacactatatagacaatttaatttttatgaaaacacatttaataatttgaggctgcaggagaataagccatcggacacaccaccagaaccatggccatgttgaaaacgacgagtcttgggttccagtaacatttccctctttaataatttctatttttctgtcaaaataattagtttttcgaaatttgaggccagaacgaccacttgtcaaatttgatttttagctgtagtaaaaacagtttgctagtgtcacagttaaccggtaattgattctttttaacgatttatagaagtaacatttttgtaaaataaaatatacattatggtatgtgacaacggaccacgcttatttgtattggtgaatcttttaattactccctccaatttattttagttgcagatttagatttatgcacatagattaataaaaatattttgcacattttcaaaataaaaacaccattacttatacaactaaccatatttcaaccaataaaaataaattagaaaatattatttataaattttgtattgaaattataaaataatacttattttaaaacgaaattaatttacaacgacaattaaactgaaacggaaagaaattattaatacttaattaaagagtttttagaaaaattgaaagacatgtttatgcgaaactcatgtgaaagtctttgaaataatagattttggtataaatatttcaaattttcttaaaataataattatatattaatataatttgtgataaaatctcgtcaaaaactcactaatgcaaatgcttttattttgaatttcttactcctctaaatgcatttacttttatactaatattattttctttctctaatttggcgtttcgtaatagtttgtctgtattttgaaaactaacaaaaaataataaaaacaaaagcttataaacacatagcatgcaatgaatatgtacgaatatatataccaatacatatctaagtactatttttccaagtacttaatcttgattactaaaattcattttaattgttcctttcagttaccagaaaggttatacaagaatttaccccacagtactcggatgctcagatacactgtccctctgcccatgctcgcttacccgatctatctggtattttttaattcctaaaatttactacaagtcattttagactgtgttttaaaacaatataattatttttgtttggttttactgcagtggtacagaagtcctggaaaagaagggtcacattttaacccatacagtggtttatttgctccaagcgagagaaagcttattgcaacttcgactacttgctggtccataatgttggcaattcttatctgtctttccttcctcgttggtccagtcacagttctcaaagtatacggtgttccttacattgtaagtttcttagtatatcataaagggtatatatttattattcaatatatatactatatgatttgtttttgtcatatatttttgaaatattcagatctttgtgatgtggttggacgctgtcacttacttgcatcaccatggtcatgatgagaagttgccttggtacagaggcaaggtaattaaattaactattacaagtattttacaaaaaactaatgattagtatatttgattaatcttaattcttgatgttttgtgattaataataggaatggagttacttacgtggaggattaacaactattgatagagattacggaattttcaacaacattcatcacgacattggaactcacgtgatccatcatcttttcccacaaatccctcactatcacttggtcgatgctgtgagtcatctcactctctggctactttcatcaaaaccatttgattaaagggtgattaattactaatgtagtgattttaacaaatggaatgtgacagacaaaagcagctaaacatgtgttgggaagatactacagagaaccaaagacgtcaggagcaataccgatccacttggtggagagtttggtagcaagtattaagaaagatcattacgtcagtgacactggtgacattgtcttctacgagactgatc

SEQ ID NO: 17 - DNA, 4676 bp, Brassica napus
aaacgtaaacaatttatacgaccacagttcgaaaataaaaacaatttatacgaccagaaatggcaaaatgttgttcttagcattttttttttaactttacttttgcgtaaaacacatttctccaatttggtttcattgcgttgaacgacgtaacaaagtaatacacctaacccttttttttggaacattatacacccaacccattgtacaaaagttacagctaaattaccctttttattcttttgataaataaaaaaataaattattaatcattaaaaaataatttggagtattttctcaatgtccatatatacatcttctccctttatataagccaacctcacacacccaaaaaatccatcaaacctttcttcaccacatttcactgaaaggccacacatctagagagagaaacttcgtccaaatctctctctccagcaatggttgttgctatggaccagcgcagcaatgttaacggagattccggtgcccggaaggaagaagggtttgatccaagcgaacaaccaccgtttaagatcggagatatcagggcggcgattcctaagcattgttgtttgaggtttaattcttttgaggttaccttttcatgttcaattattaaaaaaataaaataaaatataggatctaagatttttttcttcatcagttcaagcatcatcactcatcagtcgtaagactcgtaacaaaatatcttcttttctataattaatattatttccgcatttaatggatctacgttttgatgttctcaaattttgtttctctttctctagatccccggaacttttaattataattatagtatagtataatatcaagaaaatatactgtttattttttttggcaacaaatatattactcttgtttctttgacaagaaaaaaatatattgttttttttcttctttttgtgttccaatctattttcgagatttagacaagtgacacgtcatataccggatttgttaccttgttaaagagtttgggttaaaacaaatgtagaaaagttaaaataaattgtgcaataaatgataaatacgtttttatgttaaacaatgatgtgaaaataaaattgaataatggcagtggacatgggagtttttcagacattcctctgctgaacagtgtggttggtcacattcttcattcattcatcctcgttccttaccatggttggtaagtcatttattaactatttccatgtaaactattagtacttgttttcgtatttcttacattttcgtttgtcattcttcttgggtgcatgctagcaaactgtaatcagtattaactgggaactaccaactgttttttttttgctagagtagcaattttataattaaataagaatcctattaaacaatgcatgtgacaatatgaggttgcttttctgttcaaaacaaatctttagaagccaatgaaaaagaatccaaaacttttttttaaatgatatgcgcctatctattggtcctgactcctgagttttcttactttcttaagtataattagattttgatttttttttataggttttcactattgttatttgtttacatcagcttcagatatcttcgaaaaagatttacatgcatcaatttcatgaggatttatagtttttcttttacttatttccgacacaatgtttagtagtaaaaagcattaaatgtttttttgctcaaaaaaaaaagaatgggattgttagagcactctattgttagttgttcaataaatataccaactaaaaaaacaaaataaatataaaatgagtgagattgttaaatcattatagagacaatttcattttcacaaaaataaataaatacataactttttataattggggtttgcaggagaataagccatcggacacaccaccagaaccatggccatgttgaaaacgacgagtcttgggttccggtaatctttcctactctcgtagtttctcttgtcttttatttatttgtttgtttttcggaatttattcttatgtctatgttcttaggattctatatgtttattttattagtttatgttttcagtctgaggtcagaccgaccacttgtcagatctgttttctagctgtagtaaaaaacaatttgcaagtgtaatagttcagcataattgatcttgttagagcatttccaaaacaaactttataattttaaatatacagttttttgttctctaaaaaagaatttaaaaattttaaagtttgagggacgaaacttcaaatttgaactttcactactcaacttcaaatttgaaatttcatcttttttatttacattttgatcattataattaattatacattacatttatgattcttaagtattttctcatttattgttttaattcttaaattttttatacatcataaatatttccaatttgtttttataaattcaaattttacacaaaaaagtaataaaaattttaaataagatttataatattttaaaactataattaggcaaaaaaaatattacaaaaaaatgtaataaaaactttaaaataagatatatcaagacataattattagaaattttaaatattataacaatattaataatctggtaaatttgctccaaaacctcaaaaatttctaaattattgtccaaacaaatttgtttaaccgaatatggagcattacaaaaataattttatggaatagtgtggtattttgcttgtagttaatatttaattatgtatttctatttataattttatatatttaatgtaagatttttttaattaatattactgtaatatttttatatatgtactagttatttataaaagttttatagatttgtattagttataacaaaaataaggatcattgtgtaaaatacaaataattttgaaattacgtttaaagttttggttatgaaaaaaatactttgaaactttaaatttagagttttgcaaactttaaaatgttagatagatagtttttttggagatgcatttagtggttatggtagtaactcagaaaatgaaaaatctatacttttatactccctccgttttttaatataagtcgttttacagttatacacgtagattaagaaaaccattaatttcttatattttctagacaaaaacatcattaattatttacctaaccacaattcaaccaatataaaaatagaagatatattaccattggtcatacaacattaattattaataaattttacatagaaaaccgaaaacgacatataatttggaacaaaaaaatttctctaaaacgacttatattaaaaaacggagggagtagtacctaactttaacgatggaccacttatattcgagtccttagcataaaatgattctcctcgaaatccgtttactttcttcattattttttccttttcagttttggcgttttcgtaatacttttgtcttcaatcttgaaagctattagtataaaaacttataaacacatcacatgcaatgaattaatacgaatacataaccagaatgacaaattttcaatgaatatttaataccagtaagtactactccgtaatagtaatagtaatagtcatattaattttttttttgtcatcaaacaaacagtaatagtaatattaattataattatgtatttcagttgccagaaaagttgtacaagaacttgccccatagtactcggatgctcagatacactgttcctctgcccatgctcgcttacccgatctatctggtaaaaaaaaatacaatttcaatttttttcttaaaattacaaatggttttatattttgagttttaagccaatatataaattaattttgattggattttaactacagtggtacagaagtcctggaaaagaagggtcacattttaacccatacagtagtttatttgctccaagcgagaggaagcttattgcaacttcaacaacttgctggtccataatgttggccactcttgtttatctatcgttcctcgttggtccagtcacagttctcaaagtctatggtgttccttacattgtaagtttcacatattattacaagagatttatatattattaataataaatttgttttttgacataaagttttggaaaattttcagatctttgtaatgtggttggacgctgtcacgtacttgcatcatcatggtcacgatgagaagttgccttggtacagaggcaaggtaaataaatcaatttttaaaaagaaatgtacagaaagcaataatggttagtattgattaatcttaatttttgatgttttgcatacaataataggaatggagttatttacgtggaggattaacaactattgatagagattacggaatcttcaacaacatccatcacgacattggaactcacgtgatccatcatcttttcccacaaatccctcactatcacttggtcgatgcggtgagtgatctagctttctctctctctagtttcatttgattaaatggtgattaattactaatttaattaatgaattgtggacagacgagagcagctaaacatgtgttaggaagatactacagagagccgaagacgtcaggagcaataccgattcacttggtggagagtttggtcgcaagtattaaaaaagatcattacgtcagtgacactggtgatattgtcttctacgagacagatccagatctctacgtttat